(19) United States
(12) Patent Application Publication
(10) Pub. No.: US 2026/0306521 A1
(43) Pub. Date: Oct. 8, 2026

(54) FUCOSYLATED MAMMALIAN MILK OLIGOSACCHARIDE FOR USE IN THE PREVENTION OR TREATMENT OF PNEUMONIA

(71) Applicant: Inbiose N.V., Zwijnaarde (BE)

(72) Inventors: Joeri Beauprez, Zwijnaarde (BE); Wesley Carpentier, Zwijnaarde (BE); Ut Van Nguyen, Zwijnaarde (BE); Annelies Vercauteren, Zwijnaarde (BE); Jordi Lensink, Voorthuizen (NL); Marie Louise Miedema, Voorthuizen (NL); Mariska Van De Vosse, Voorthuizen (NL); Elian Elisabeth Marie-Louise Verscheijden, Voorthuizen (NL)

(21) Appl. No.: 19/480,740

(22) PCT Filed: May 3, 2024
(86) PCT No.: PCT/EP2024/062260

§ 371 Date: Oct. 31, 2025

(30) Foreign Application Priority Data
May 4, 2023 (EP) 23171610.1
May 4, 2023 (LU) LU504126
Jun. 14, 2023 (EP) 23179353.0

(51) Int. Cl. A61K 31/702; A61P 11/00

(52) U.S. Cl. CPC A61K 31/702; A61P 11/00

(57) ABSTRACT

The present invention relates to a fucosylated mammalian milk oligosaccharide (MMO) for use in a method for preventing and/or treating pneumonia in a farmed animal or a domesticated animal.

### FIELD OF THE INVENTION

The present invention relates to a fucosylated mammalian milk oligosaccharide (MMO) for use in a method for preventing and/or treating pneumonia in a farmed animal or a domesticated animal.

### BACKGROUND OF THE INVENTION

The health status of a farmed animal, i.e. an animal that is reared in an agricultural setting in order to produce various commodities such as food, hair or wool, has a tremendous impact on the economic endurance of farms due to the medical costs, animal losses and the long-term effects on the performance. In contrast to the natural way, farmed animals such as bovine are separated from their dams already within a short timeframe (hours or a few days) after they are born for commercial activities and practical reasons. This goes together with a high chance of ingesting germs upon separation. A significant disease affecting farmed animals but also domesticated animals is pneumonia which causes inflammation of the lung tissue and airways. It is unfortunately the most common reason for death and poor performance in these animals (Poulsen and Mcguirk, 2009, Vet. Clin. North. Am. Food anim. Pract. 25 (1): p. 121-137; Arne et al, 2011, Int J. Microbiol., article 746356). Pneumonia can be caused by viruses, bacteria, fungi, parasites and protozoa.

Moreover, farmed animals are typically transported to rearing facilities. This transport not only brings animals close together (sometimes from different farms together), but it is well-known that such stressful transportation has negative effects on immunity, response to infection and inflammatory responses. In these animals, shipping fever may occur which may eventually lead to pneumonia as well as well-documented for e.g. bovine and horse (Earley et al, 2017, Animal 11 (3): p. 486-492; Vivek Joshi et al, 2016, J. Immunol. And Immunopath. 18 (2)).

The development of an effective therapeutic for preventing and/or treating pneumonia is a medical and economical imperative.

### SUMMARY OF THE INVENTION

It was surprisingly found that a fucosylated MMO according to the invention proves to be an efficacious agent to prevent and/or treat pneumonia in a farmed animal or a domesticated animal, in particular in animals which are bred in larger groups. Said fucosylated MMO is not associated with adverse effects and is safe to use in animals.

In a first aspect, the invention provides a fucosylated MMO for use in a method for preventing and/or treating pneumonia in an animal, wherein said animal is a farmed animal or a domesticated animal.

In a second aspect, the invention provides a method for preventing and/or treating pneumonia in a farmed animal or a domesticated animal, wherein a fucosylated MMO according to the first aspect is administered to said animal.

In a third aspect, the invention provides the use of a fucosylated MMO according to the first aspect for the manufacture of a medicament for preventing and/or treating pneumonia in a farmed animal or a domesticated animal.

### DETAILED DESCRIPTION OF THE INVENTION

### Fucosylated MMO for Use in Preventing/Treating Pneumonia

In a first aspect, the invention provides a fucosylated mammalian milk oligosaccharide (MMO) for use in a method for preventing and/or treating pneumonia in an animal, wherein said animal is a farmed animal or a domesticated animal, preferably a farmed animal.

Preferably, the invention provides a composition comprising a fucosylated MMO, wherein said composition is for use in a method for preventing and/or treating pneumonia in an animal, wherein said animal is a farmed animal or a domesticated animal, preferably a farmed animal. Throughout the application and claims, the term “a fucosylated MMO” is preferably replaced with “a composition comprising a fucosylated MMO”, more preferably replaced with “a composition consisting essentially of a fucosylated MMO”, even more preferably replaced with “a composition consisting of a fucosylated MMO”.

### Pneumonia

In an embodiment of the first aspect of the invention, said fucosylated MMO is for use in a method for preventing and/or treating pneumonia in an animal, wherein said animal is a farmed animal or a domesticated animal, preferably a farmed animal.

In a preferred embodiment, said pneumonia is acute pneumonia. As understood by the skilled person, acute pneumonia entails inflammation of the alveoli and interstitial tissues of the lungs resulting in acute respiratory symptoms and signs including cough, breathlessness, chest pain and sputum production (Torres et al, 2021, Nature Reviews Disease primers 7 (25); Ames, 2016, Vet. Clin. North Am. Food Anim. Pract. 13 (3): p. 379-391). The alveoli are filled with pus and fluid (in contrast to a healthy subject wherein the alveoli are filled with air when breathing) which makes breathing painful and limits oxygen intake. Pneumonia is caused by a number of infectious agents, including viruses, bacteria and fungi. Chronic pneumonia is pneumonia that lasts at least 6 weeks; a radiograph of the chest usually shows diffuse or focal shadows.

In an additional and/or alternative preferred embodiment, said pneumonia is shipping fever pneumonia. Shipping fever is a pulmonary disorder that is well-described and occurs in animals which are transported over long distances. Shipping fever has a multifactorial aetiology of infectious agents (viral and/or bacterial; usually a primary viral infection is followed by a secondary bacterial superinfection), environment stress factors and host factors (Earley et al, 2017, Animal 11 (3): p. 486-492; Gaudino et al, 2022, Vet. Res. 53 (1); Maeda and Oikawa, 2019, Front. Vet. Sci. 6 (27)).

In this context of the invention, said animal is preferably a horse or a bovine, more preferably a bovine. When said animal is a bovine, than “shipping fever” is also referred to as “bovine respiratory disease (BRD)” or “bovine respiratory disease complex” (Vivek Joshi et al, 2016, J. Immunol. And Immunopath. 18 (2); Earley et al, 2017, Animal 11 (3): p. 486-492). Hence, when said animal is a bovine, the terms “shipping fever”, “bovine respiratory disease”, “BRD” and “bovine respiratory disease complex” can be interchangeably used in the context of the present invention.

In additional and/or alternative preferred embodiment, said pneumonia is brooder pneumonia. As understood by the skilled person, brooder pneumonia is a disease that results in pneumonia in poultry, especially in chicks. “Brooder pneumonia” is also referred to as “Aspergillosis” and these terms can hence be interchangeably used in the context of the present invention. Clinical symptoms include respiratory distress (dyspnea and gasping), central nervous dysfunction (tremors, ataxia and torticollis), somnolence (sleepy), inappetence and emaciation. Causative agent include Aspergillus fumigatus, Aspergillus flavus and Aspergillus niger.

In this context of the invention, said animal is preferably poultry, more preferably a chicken, a quail, a turkey, a duck, a pigeon and a goose, even more preferably a chicken, a quail or a turkey, even more preferably a chicken or a quail, most preferably a chicken. It is preferred that the animal is a young animal, more preferably a chick. Throughout the application and claims, the term “chicken” is preferably replaced with broiler.

In the context of the present invention, said pneumonia is preferably not caused by SARS-COV-2, more preferably is not caused by a virus resulting in coronavirus disease (e.g. covid-19).

The inventors surprisingly found that a fucosylated mammalian milk oligosaccharide (MMO) according to the invention enhances the immune response in the treated subject, preferably enhances the T lymphocyte immunity in the treated subject, more preferably enhances the T lymphocyte activation in the treated subject.

The technical effect of treating a subject with a fucosylated MMO according to the invention hence encompasses an indirect mechanism of protection by stimulating the immune response in the treated subject as described herein to protect and/or fight off pathogens, rather than a direct mechanism wherein the fucosylated MMO would directly act on the pathogen and/or would block its entry by binding to an essential receptor for the pathogen.

This technical effect can be assessed in a subject by the skilled person by using routinely available techniques in the art. A particularly useful and preferred method is flow cytometry (preferably fluorescence-activated cell sorting, i.e. FACS) to assess the activation of T cells of a subject. Herein, peripheral blood mononuclear cells can be isolated from a subject's blood sample using standard techniques such as density gradient centrifugation. To identify activated T lymphocytes, a panel of suitable activated T cell markers can be used during flow cytometry (preferably FACS) analysis. This includes the classical T cell markers such as CD3, CD4 and CD8, combined with one or more known activation markers such as CD69, CD25 and HLA-DR. An alternative method to assess said technical effect is illustrated in the Examples below (differential expression analysis of whole transcriptome).

### Subject

In an embodiment of the first aspect of the invention, said fucosylated MMO is for use in a method for preventing and/or treating pneumonia in an animal, wherein said animal is a farmed animal or a domesticated animal, preferably a farmed animal.

As understood by the skilled person, a “farmed animal” refers to an animal that is reared in an agricultural setting in order to produce various commodities such as food (meat, organs, eggs, dairy products) and/or hair or wool. A farmed animal is preferably selected from the list consisting of bovine, pig, sheep, goat, horse, donkey, mule, hinny, camelid (preferably llama or alpaca), rabbit and poultry: more preferably selected from the list consisting of bovine, pig, sheep, goat, horse, camelid (preferably llama or alpaca), rabbit and poultry; even more preferably selected from the list consisting of bovine, pig, sheep, goat, horse, camelid (preferably llama or alpaca) and poultry; even more preferably selected from the list consisting of bovine, pig, sheep, goat, camelid (preferably llama or alpaca) and poultry; even more preferably selected from the list consisting of bovine, pig, sheep, goat, and poultry; even more preferably selected from the list consisting of bovine, pig, sheep and goat; even more preferably selected from the list consisting of bovine, sheep and goat; most preferably said farmed animal is a bovine.

Alternatively, a farmed animal is preferably selected from the list consisting of bovine and pig, most preferably said farmed animal is a bovine.

As understood by the skilled person, a “domesticated animal” refers to an animal that has been selectively bred and adapted over generations to live alongside humans. A domesticated animal in the context of the present invention is preferably a companion animal, a horse, a donkey, a mule or a hinny; more preferably said domesticated animal is selected from the list consisting of a dog, a cat, a horse, a donkey, a mule and a hinny; even more preferably said domesticated animal is selected from the list consisting of a dog, a cat and a horse; most preferably said domesticated animal is a horse.

In the context of the present invention, the term “poultry” refers to birds bred by humans for their eggs, meat and/or feathers. Throughout the application and claims, said poultry preferably is a chicken, a quail, a turkey, a duck, a pigeon and a goose, more preferably a chicken, a quail or a turkey, more preferably chicken or a quail, most preferably a chicken. Throughout the application and claims, the term “chicken” is preferably replaced with broiler.

In a preferred embodiment of the invention, said farmed animal or domesticated animal is selected from the list consisting of bovine, pig, sheep, goat, horse, donkey, mule, hinny, camelid (preferably llama or alpaca), dog, cat, rabbit and poultry; more preferably selected from the list consisting of bovine, pig, sheep, goat, horse, donkey, mule, hinny, camelid (preferably llama or alpaca) and poultry; even more preferably selected from the list consisting of bovine, sheep, goat, horse, donkey, mule, hinny, camelid (preferably llama or alpaca) and poultry; even more preferably selected from the list consisting of bovine, horse, donkey, mule, hinny and poultry; even more preferably selected from the list consisting of bovine, horse, donkey, mule and hinny; even more preferably selected from the list consisting of bovine and horse; most preferably said animal is bovine.

In an additional and/or alternative preferred embodiment, said animal is a multigastric animal, preferably a ruminant or camelid, more preferably a ruminant.

Preferably, said camelid is a llama or an alpaca, more preferably said camelid is an alpaca.

Preferably, said ruminant is selected from the list consisting of bovine, sheep and goat, more preferably said ruminant is bovine.

In an alternative preferred embodiment, said animal is a monogastric animal, preferably selected from the list consisting of pig, horse, donkey, mule, hinny, rabbit and poultry, more preferably selected from the list consisting of pig, horse, donkey, mule, hinny and poultry, even more preferably selected from the list consisting of pig, horse, donkey, mule and hinny, even more preferably pig or horse, most preferably pig.

Throughout the description and claims, unless specifically stated otherwise, the terms “subject” and “animal” can be preferably replaced with “subject in the need thereof” and “animal in the need thereof”, respectively.

In a more preferred embodiment, said animal according to the invention is a non-adult animal. In an additional and/or alternative more preferred embodiment, said animal according to the invention is a young animal. Throughout the application and claims, unless specifically stated otherwise, a “non-adult animal” can be preferably replaced with “juvenile animal” or “young animal”.

As understood by the skilled person:
a non-adult bovine is a calf;
a non-adult pig is a piglet;
a non-adult sheep is a lamb;
a non-adult goat is a kid;
a non-adult horse is a foal;
a non-adult llama is a cria;
a non-adult alpaca is a cria;
a non-adult dog is a puppy;
a non-adult cat is a kitten;
a non-adult rabbit is a kitten or a kit;
a non-adult poultry/chicken is a chick.

In an additional and/or alternative more preferred embodiment, said animal according to the invention is a preweaned animal.

In an additional and/or alternative more preferred embodiment, said animal according to the invention is an animal that is weaned.

As understood by the skilled person, weaning refers to the process of withdrawing the access of a young animal to milk and to gradually accustom the young animal to the adult diet.

Throughout the application and claims, each embodiment disclosed in the context wherein the subject is a horse, is also disclosed for a subject being a donkey, a mule or a hinny.

Method for Preventing and/or Treating Pneumonia

In an embodiment of the first aspect of the invention, said fucosylated MMO is for use in a method for preventing and/or treating pneumonia in an animal, wherein said animal is a farmed animal or a domesticated animal, preferably a farmed animal.

Said animal is as described in the Section “Subject”.

In the context of the present invention, the term “preventing” pneumonia as described herein, preferably means avoiding that said pneumonia occurs and/or decreasing the incidence of said pneumonia. In other words, “preventing” preferably refers to ameliorating the risk of suffering from said pneumonia. The term “prevention” and “prophylaxis” are interchangeably used in the context of the present invention. The term “treating” pneumonia as described herein, preferably means inhibiting said pneumonia, e.g. arresting the development of said pneumonia; relieving said pneumonia, for example causing regression of said pneumonia; and/or relieving a condition caused by or resulting from said pneumonia, e.g. relieving, preventing or treating symptoms of said pneumonia. In other words, “treating” preferably refers to decreasing the duration (number of days/weeks/months) the subject will suffer from said pneumonia), the risks, the complications and/or the severity of said pneumonia; this also encompasses the relief of the symptoms caused by said pneumonia.

In a preferred embodiment, said method according to the invention comprises administering an effective amount of said fucosylated MMO (it is referred to the Section “Oligosaccharide”) according to the invention to said animal (it is referred to the Section “Subject”). As understood by the skilled person, an “effective amount” is the amount of said oligosaccharide which is required to confer a therapeutic effect on the animal as described in the present application. Effective amounts vary, as recognized by those skilled in the art, depending on the subject, route of administration, excipient usage among other known factors.

In an additional and/or alternative preferred embodiment, said fucosylated MMO is administered to said animal at least once, preferably at least twice, between birth (0 weeks) and 5 weeks of age, preferably between birth and 4 weeks of age, more preferably between birth and 3 weeks of age.

In an additional and/or alternative preferred embodiment, said fucosylated MMO is administered to said animal at least once, preferably at least twice, more preferably daily:
in the three weeks, preferably two weeks, more preferably one week, preceding the day of transporting said animal; and/or
on the day of transporting said animal; and/or
in the three weeks, preferably two weeks, more preferably one week, after the day of transporting said animal.

More preferably said fucosylated MMO is administered to said animal at least once, preferably at least twice, more preferably daily:
(i)
   in the three weeks, preferably two weeks, more preferably one week, preceding the day of transporting said animal; and/or
   on the day of transporting said animal;
      or
(ii)
   on the day of transporting said animal; and/or
   in the three weeks, preferably two weeks, more preferably one week, after the day of transporting said animal.

In a more preferred embodiment, said fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, said fucosylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, said fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g. In the context of the present invention, an amount of an oligosaccharide according to the invention expressed in a number of grams or milligrams per daily dose as used herein means that the amount of the oligosaccharide is such that when administering the daily dosage to a subject, the subject will be administered with the number of grams or milligrams of the oligosaccharide. In other words, if the daily dosage is for example 50 mg, then the subject receives in total 50 mg per day. This may be in one or more portions. So, if the daily dosage is 50 mg divided over 2 portions, then a single serving consists of 25 mg, a daily serving consists of 2 of such single servings.

In an additional and/or alternative more preferred embodiment, said fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of said animal.

In an even more preferred embodiment, said fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g. This embodiment is for example particularly preferred if said animal is a bovine or a horse, more preferably wherein said animal is a calf or a foal, most preferably wherein said animal is a calf.

In an additional and/or alternative even more preferred embodiment, said fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of said animal. This embodiment is for example particularly preferred if said animal is a bovine or a horse, more preferably wherein said animal is a calf or a foal, most preferably wherein said animal is a calf.

In an alternative even more preferred embodiment, said fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.

In an additional and/or alternative even more preferred embodiment, said fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of said animal. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.

In an even more preferred embodiment, said fucosylated MMO is administered to said animal for at least 1 week, preferably at least 2 weeks, more preferably at least 3 weeks, even more preferably at least 4 weeks, most preferably at least 6 weeks.

In an additional and/or alternative even more preferred embodiment, said fucosylated MMO is administered to said animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.

In an even more preferred embodiment, said fucosylated MMO is administered to said animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.

In an even more preferred embodiment, said fucosylated MMO is administered to said animal for:
if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
if subject is a pig: 1-18, preferably 1-12, more preferably 1-8, even more preferably 1-6, even more preferably 1-4, most preferably 2-4, consecutive weeks;
if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
if subject is a horse: 1-18, preferably 1-12, more preferably 1-8, even more preferably 1-6, even more preferably 1-4, most preferably 2-4, consecutive weeks;
if subject is a camelid: 1-18, preferably 1-12, more preferably 1-8, even more preferably 1-6, even more preferably 1-4, most preferably 2-4, consecutive weeks;
if subject is a rabbit: 1-12, preferably 1-8, more preferably 1-6, even more preferably 1-4, most preferably 2-4, consecutive weeks;
if subject is a dog: 1-12, preferably 1-8, more preferably 1-6, even more preferably 1-4, most preferably 2-4, consecutive weeks;
if subject is a cat: 1-12, preferably 1-8, more preferably 1-6, even more preferably 1-4, most preferably 2-4, consecutive weeks;
if subject is poultry: 1-8, preferably 1-6, more preferably 1-4, most preferably 2-4, consecutive weeks.

Preferably, said fucosylated MMO according to the invention is administered at least once a week (i.e. at least one daily dosage is administered in a week), more preferably at least once every 3 days, even more preferably at least once every 2 days, most preferably at least once daily (i.e. a daily dosage is administered every day).

The inventors have surprisingly found that a fucosylated MMO according to the invention is sufficient to obtain an effective therapeutic for preventing and/or treating pneumonia as described herein. The administration of other (oligo) saccharides is not required to obtain said therapeutic effect. In this context of the invention, the invention provides a fucosylated MMO and a sialylated MMO, preferably a fucosylated MMO and two or more sialylated MMOs (i.e. a first sialylated MMO and a second sialylated MMO, optionally further sialylated MMO's are administered), more preferably a fucosylated MMO and two sialylated MMO's (i.e. a first sialylated MMO and a second sialylated MMO), for use in a method for preventing and/or treating pneumonia in an animal, wherein said animal is a farmed animal or a domesticated animal, preferably a farmed animal. For the sake of clarity, the expression “more preferably two sialylated MMO's” in this context of the invention means that no additional sialylated MMO's are administered to the subject. In other words, said method for preventing and/or treating pneumonia in an animal as described herein comprises administering a fucosylated MMO as described herein and a sialylated MMO, preferably a fucosylated MMO and two or more sialylated MMOs (i.e. a first sialylated MMO and a second sialylated MMO, optionally further sialylated MMO's are administered), more preferably a fucosylated MMO and two sialylated MMO's (i.e. a first sialylated MMO and a second sialylated MMO).

It is preferred that the administered MMO's are administered as a mixture, more preferably as part of a composition, even more preferably as part of a composition according to Section “Composition”. Said fucosylated MMO is as described herein (it is referred to the Section “Oligosaccharide). In a preferred embodiment, said sialylated MMO comprises a sialic acid, preferably a Neu5Ac, that is linked to a monosaccharide in an alpha-2,3-, alpha-2,6- or alpha-2,8-linkage, preferably an alpha-2,3- or an alpha-2,6-linkage, and wherein said monosaccharide is preferably selected from galactose, N-acetylglucosamine and Neu5Ac, more preferably said monosaccharide is galactose or N-acetylglucosamine, even more preferably said monosaccharide is galactose. The skilled person will understand that the expression “sialic acid is linked to a monosaccharide” refers to the situation wherein the sialic acid is bound to a monosaccharide through a glycosidic bond and wherein said sialic acid and monosaccharide are part of the oligosaccharide of the invention (which can comprise additional monosaccharide(s) than said sialic acid and said monosaccharide). In an additional and/or alternative preferred embodiment, said sialylated MMO consists of 3-8, more preferably 3-7, even more preferably 3-6, most preferably 3-5, monosaccharides. In an additional and/or alternative preferred embodiment, said sialylated MMO comprises a lactose, a lacto-N-biose (LNB) or N-acetyllactosamine (LacNAc) at its reducing end, preferably said sialylated MMO comprises lactose or LacNAc at is reducing end, most preferably said sialylated MMO comprises lactose at its reducing end.

A sialylated MMO comprising lactose at its reducing end is preferably selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c and LST d; more preferably selected from the list consisting of 3′SL, 6′SL, 3,6-disialyllactose, 6,6′-disialyllactose, 8,3-disialyllactose, LST a, LST b, LST c and LST d; even more preferably selected from the list consisting of 3′SL, 6′SL, LST a, LST b, LST c and LST d; most preferably wherein said sialylated MMO is 3′-sialyllactose (3′SL) or 6′-sialyllactose (6′SL). A sialylated MMO comprising lacto-N-biose (LNB) at its reducing end is preferably selected form the list consisting of 3′-sialyllacto-N-biose (3′SLNB), 6′-sialyllacto-N-biose (6′SLNB) and monofucosylmonosialyllacto-N-octaose (sialyl Lewis a); more preferably said sialylated MMO is 3′SLNB or 6′SLNB. A sialylated MMO comprising N-acetyllactosamine (LacNac) at its reducing end is preferably selected form the list consisting of 3′-sialyllactosamine (3′SLacNAc), 6′-sialyllactosamine (6′SLacNAc) and sialyl Lewis x; more preferably said sialylated MMO is 3′SLacNAc or 6′SLacNAc. In a more preferred embodiment, said sialylated MMO according to the invention is selected from a list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. More preferably said sialylated MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said sialylated MMO is selected from the list consisting of 3′SL, 6′SL, 3′SLNB, 6′SLNB, 3′SLacNAc and 6′SLacNAc. Most preferably said sialylated MMO is 3′-sialyllactose (3′SL) or 6′-sialyllactose (6′SL). In an additional and/or alternative more preferred embodiment, said first sialylated MMO according to the invention is selected from a list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. Even more preferably said first sialylated MMO is selected from the list consisting of 3′SL, 6′SL, 3'S-2′FL, 6'S-2′FL, 3'S-3-FL, 6'S-3-FL, LST a, LST b, LST c, LST d, 3′SLNB, 6′SLNB, sialyl Lewis a, 3′SLacNAc, 6′SLacNAc and sialyl Lewis x. Even more preferably said first sialylated MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said first sialylated MMO is selected from the list consisting of 3′SL, 6′SL, 3′SLNB, 6′SLNB, 3′SLacNAc and 6′SLacNAc. Even more preferably said first sialylated MMO is 3′-sialyllactose (3′SL) or 6′-sialyllactose (6′SL), most preferably said first sialylated MMO is 3′SL. In an additional and/or alternative more preferred embodiment, said second sialylated MMO according to the invention is selected from a list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), monofucosylmonosialyllacto-N-octaose (sialyl Lewis a), 3′-sialyllactosamine (3′SLacNAc), 6′-sialyllactosamine (6′SLacNAc) and sialyl Lewis x. Even more preferably said second sialylated MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said second sialylated MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said second sialylated MMO is selected from the list consisting of 3′SL, 6′SL, 3′SLNB, 6′SLNB, 3′SLacNAc and 6′SLacNAc. Even more preferably said second sialylated MMO is 6′-sialyllactose (6′SL) or 3′-sialyllactose (3′SL), most preferably said second sialylated MMO is 6′SL.

When a fucosylated MMO and a first sialylated MMO are administered to said animal, said fucosylated MMO and said first sialylated MMO are preferably administered on the same day, more preferably administered simultaneously. Further, it is a preferred embodiment that the amount of said first sialylated MMO administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal. Further, it is a more preferred embodiment that the amount of said fucosylated MMO administered to said animal is lower than the amount of said first sialylated MMO, preferably said amount of said fucosylated MMO is 1.1 to 10.0, preferably 1.5-7.5, more preferably 2.5-7.5, most preferably 4.0-7.0, times lower than the amount of said first sialylated MMO.

When a fucosylated MMO, a first sialylated MMO and a second sialylated MMO according to the invention are administered to said animal, said fucosylated MMO, said first sialylated MMO and said second sialylated MMO are preferably administered on the same day, more preferably administered simultaneously. Further, it is a preferred embodiment that the ratio between the amount of said first sialylated MMO administered to said animal and the amount of said second sialylated MMO administered to said animal is similar to this ratio as found in the mother's milk of the same species (preferably as described in the Section “Composition”). More preferably, the amount of said first sialylated MMO administered to said animal is higher than the amount of said second sialylated MMO, preferably said amount of said first sialylated MMO is 1.1 to 40.0, more preferably 1.1 to 35.0, most preferably 1.5 to 35.0, times higher than the amount of said second sialylated MMO. Even more preferably, the amount of said first sialylated MMO administered to said animal is higher than the amount of said second sialylated MMO, preferably said amount of said first sialylated MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of said second sialylated MMO. It is an additional and/or alternative preferred embodiment that the amount of said first sialylated MMO administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal. Preferably, the amount of said fucosylated MMO administered to said animal is lower than the amount of said first sialylated MMO, preferably said amount of said fucosylated MMO is 1.1 to 10.0, preferably 1.5-7.5, more preferably 2.5-7.5, most preferably 4.0-7.0, times lower than the amount of said first sialylated MMO. It is an additional and/or alternative preferred embodiment that the amount of said second sialylated MMO administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal. Preferably, the amount of said fucosylated MMO administered to said animal is lower than the amount of said second sialylated MMO, preferably said amount of said fucosylated MMO is 1.1 to 10.0, preferably 1.5-7.5, more preferably 1.5-5.0, most preferably 2.5-5.0, times lower than the amount of said second sialylated MMO.

It is a more preferred embodiment that the total amount of both first sialylated MMO and second sialylated MMO is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal. Preferably, the amount of said fucosylated MMO administered to said animal is lower than the total amount of both first sialylated MMO and second sialylated MMO, preferably said amount of said fucosylated MMO is 1.1 to 15.0, preferably 1.5 to 15.0, more preferably 2.5 to 15.0, even more preferably 5.0 to 15.0, even more preferably 5.0 to 12.5, most preferably 5.0 to 10.0, times lower than the total amount of both first sialylated MMO and second sialylated MMO. It is an even more preferred embodiment that the amount of all sialylated MMOs that are administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated administered to said animal. Preferably, the amount of said fucosylated MMO administered to said animal is lower, preferably 1.1 to 15.0 times lower, more preferably 1.5 to 15.0 times lower, even more preferably 2.5 to 15.0 times lower, even more preferably 5.0 to 15.0 times lower, even more preferably 5.0 to 12.5 times lower, most preferably 5.0 to 10.0 times lower, than the amount of all sialylated MMOs administered to said animal.

In this context of the invention, said sialylated MMO/first sialylated MMO/second sialylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g. More preferably, said sialylated MMO/first sialylated MMO/second sialylated MMO is administered at a daily dose of 0.001-15.0 g, preferably 0.001-10.0 g, more preferably 0.001-5.0 g, even more preferably 0.001-2.0 g, even more preferably 0.001-1.0 g, even more preferably 0.005-1.0 g, most preferably 0.010-1.0 g. Even more preferably, said sialylated MMO/first sialylated MMO/second sialylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.

In an additional and/or alternative more preferred embodiment, said sialylated MMO/first sialylated MMO/second sialylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of said animal.

In an even more preferred embodiment, said sialylated MMO/first sialylated MMO is administered at a daily dose of 0.01-1.0 g, preferably 0.01-0.75 g, more preferably 0.025-0.75 g, even more preferably 0.05-0.75 g, most preferably 0.05-0.50 g. This embodiment is for example particularly preferred if said animal is a bovine or a horse, more preferably wherein said animal is a calf or a foal, most preferably wherein said animal is a calf. In an additional and/or alternative even more preferred embodiment, said sialylated MMO/first sialylated MMO is administered at a daily dose of 0.1-100.0 mg, preferably 0.1-75.0 mg, more preferably 0.5-75.0 mg, even more preferably 1.0-75.0 mg, even more preferably 2.5-75.0 mg, most preferably 2.5-50.0 mg, per kg bodyweight of said animal. This embodiment is for example particularly preferred if said animal is a bovine or a horse, more preferably wherein said animal is a calf or a foal, most preferably wherein said animal is a calf.

In an alternative even more preferred embodiment, said sialylated MMO/first sialylated MMO is administered at a daily dose of 0.01-5.0 g, preferably 0.1-2.5 g, more preferably 0.1-2.0 g, even more preferably 0.1-1.5 g, even more preferably 0.2-1.5 g, even more preferably 0.3-1.5 g, even more preferably 0.4-1.5 g, most preferably 0.5-1.5 g. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet. In an additional and/or alternative even more preferred embodiment, said sialylated MMO/first sialylated MM is administered at a daily dose of 0.1-150.0 mg, preferably 0.5-150.0 mg, more preferably 1.0-150.0 mg, even more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 50.0-125.0 mg, most preferably 75.0-125.0 mg, per kg bodyweight of said animal. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.

In an additional and/or alternative even more preferred embodiment, said second sialylated MMO is administered at a daily dose of 0.01-1.0 g, preferably 0.01-0.75 g, more preferably 0.025-0.75 g, even more preferably 0.025-0.50 g, most preferably 0.025-0.25 g. This embodiment is for example particularly preferred if said animal is a bovine or a horse, more preferably wherein said animal is a calf or a foal, most preferably wherein said animal is a calf. In an additional and/or alternative even more preferred embodiment, said second sialylated MMO is administered at a daily dose of 0.1-100.0 mg, preferably 0.1-75.0 mg, more preferably 0.25-75.0 mg, even more preferably 0.5-75.0 mg, even more preferably 1.0-75.0 mg, even more preferably 1.0-50.0 mg, most preferably 1.0-25.0 mg, per kg bodyweight of said animal. This embodiment is for example particularly preferred if said animal is a bovine or a horse, more preferably wherein said animal is a calf or a foal, most preferably wherein said animal is a calf.

In an alternative even more preferred embodiment, said second sialylated MMO is administered at a daily dose of 0.01-5.0 g, preferably 0.1-2.5 g, more preferably 0.1-2.0 g, even more preferably 0.1-1.5 g, even more preferably 0.1-1.2 g, even more preferably 0.15-1.2 g, even more preferably 0.2-1.2 g, most preferably 0.25-1.2 g. This embodiment is for example particularly preferred if said animal is a pig, a sheep or a goat, more preferably wherein said animal is a piglet, a lamb or a kid, most preferably wherein said animal is a piglet. In an additional and/or alternative even more preferred embodiment, said second sialylated MMO is administered at a daily dose of 0.1-150.0 mg, preferably 1.0-150.0 mg, more preferably 1.0-125.0 mg, even more preferably 2.5-125.0 mg, even more preferably 5.0-125.0 mg, even more preferably 10.0-125.0 mg, even more preferably 25.0-125.0 mg, even more preferably 25.0-100.0 mg, most preferably 25.0-80.0 mg, per kg bodyweight of said animal. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.

While additional (oligo) saccharides can be administered according to the invention, the present invention relates to the fact that only the fucosylated MMO according to the invention is required for achieving an efficient therapeutic effect as described herein.

It is hence particularly preferred that said method for preventing and/or treating pneumonia in an animal as described herein, does not comprise administering lacto-N-tetraose (LNT). In an additional and/or alternative preferred embodiment, said method for preventing and/or treating pneumonia in an animal as described herein, does not comprise administering lacto-N-neotetraose (LNnT). In an additional and/or alternative preferred embodiment, said method for preventing and/or treating pneumonia in an animal as described herein, does not comprise administering N-acetyllactosamine.

In an additional and/or alternative preferred embodiment, said method for preventing and/or treating pneumonia in an animal as described herein, does not comprise administering an oligosaccharide containing N-acetyllactosamine, preferably does not comprise a saccharide containing N-acetyllactosamine. In an additional and/or alternative preferred embodiment, said method for preventing and/or treating pneumonia in an animal as described herein, does not comprise administering 3′sialyllactose. In an additional and/or alternative preferred embodiment said method for preventing and/or treating pneumonia in an animal as described herein, does not comprise administering 6′sialyllactose. More preferably, said method for preventing and/or treating pneumonia in an animal as described herein, does not comprise administering a sialylated mammalian milk oligosaccharide, even more preferably does not comprise administering a sialylated oligosaccharide, most preferably does not comprise administering a sialylated saccharide. In an additional and/or alternative preferred embodiment, said method for preventing and/or treating pneumonia in an animal as described herein, does not comprise administering a fructo-oligosaccharide (FOS). In an additional and/or alternative preferred embodiment, said method for preventing and/or treating pneumonia in an animal as described herein, does not comprise administering a galactose-oligosaccharide (GOS). In an additional and/or alternative preferred embodiment, said method for preventing and/or treating pneumonia in an animal as described herein, does not comprise administering an isomaltooligosaccharide.

In an even more preferred embodiment, said method for preventing and/or treating pneumonia in an animal as described herein, does not comprise administering a neutral non-fucosylated MMO. In an even more preferred embodiment, said method for preventing and/or treating pneumonia in an animal as described herein, comprises administering a fucosylated MMO according to the invention and no additional MMOs (preferably no additional oligosaccharides, more preferably no additional saccharides). In an even more preferred embodiment, said method for preventing and/or treating pneumonia in an animal as described herein, only comprises administering the fucosylated MMO as described herein. In another preferred embodiment, said method for preventing and/or treating pneumonia in an animal as described herein further comprises administering a microbial strain which does not use, preferably is not capable to use, said fucosylated MMO as carbon source. In an alternative preferred embodiment, said method for preventing and/or treating pneumonia in an animal as described herein does not comprise administering a microbial strain.

### Oligosaccharide

A fucosylated mammalian milk oligosaccharide (MMO) according to the invention is a MMO comprising a fucose.

As understood by the skilled person, mammalian milk oligosaccharides (MMOs) comprise oligosaccharides present in milk found in any phase during lactation including colostrum milk from humans (i.e. human milk oligosaccharides or HMOs) and mammals including but not limited to cows (Bos taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus) (Urashima T. et al., 2011, Milk Oligosaccharides, Nova Biomedical Books, New York ISBN 978-1-61122-831-1; Coppa et al, 2013, Ital. J. Pediatr. 2013, 39 (2)). A replete amount of milk saccharide structures have been elucidated so far. The majority of milk oligosaccharides found in animals such as mammals and humans comprise lactose at the reducing end (Urashima et al, 2011). Other milk oligosaccharides comprise N-acetyllactosamine (Gal-β1,4-GlcNAc) or lacto-N-biose (Gal-β1,3-GlcNAc) at the reducing end (Urashima et al, 2011; Wrigglesworth et al, 2020, PLOS ONE 15 (12); Urashima et al, 2013, Biosci. Biotechnol. Biochem 77 (3): p. 455-466; Wei et al, 2018, Sci. Rep. 8:4688). Examples hereof are 3-FLN (Gal-β1,4-(Fuc-α1,3-) GlcNAc; also known as Lewis×antigen), 3′-SLN (Neu5Ac-α2,3-Gal-β1,4-GlcNAc), 6′-SLN (Neu5Ac-α2,6-Gal-β1,4-GlcNAc) (Urashima et al, 2011; Wrigglesworth et al, 2020; Wei et al, 2018). Further, milk saccharides comprise milk glycosaminoglycans (GAGs; Coppa et al, 2013; Rai et al, 2021, Int. J. Biol. Macromolecules, 193 (A): p. 137-144). Antigens of the human ABO blood group system such as the A determinant (GalNAc-alpha1,3 (Fuc-alpha1,2)-Gal), B determinant (Gal-alpha1,3 (Fuc-alpha1,2)-Gal) and H-determinant (Fuc-alpha1,2-Gal) have been shown to be present in saccharides of the milk of mammals (e.g. A tetrasaccharide in a lion and leopard; Wrigglesworth et al, 2020).

Throughout the application and claims, a “mammalian milk oligosaccharide (MMO)” is preferably a “human milk oligosaccharide (HMO)”.

In the context of the present invention, the term “oligosaccharide” preferably refers to a saccharide containing 2 up to and including 20 monosaccharides, i.e. the degree of polymerization (DP) is 2-20, more preferably refers to a saccharide containing 3 up to and including 20 monosaccharides, i.e. the degree of polymerization (DP) is 3-20. In on other words, the term “oligosaccharide” preferably refers to a saccharide consisting of 2-20, more preferably 3-20, monosaccharide units which are linked to each other via glycosidic bonds in a linear or in a branched structure. The linkage (i.e. glycosidic linkage), between two sugar units, i.e. two monosaccharides, can be expressed, for example, as 1,4, 1->4, or (1-4), which are used interchangeably herein.

More preferably, an oligosaccharide as described herein consists of 3-8, even more preferably 3-7, even more preferably 3-6, most preferably 3-5, monosaccharides. For the sake of clarity, the expression “x-y” refers to a range from and including x to and including y. For example, 3-5 monosaccharides means that 3, 4 or 5 monosaccharides are present.

Preferably, said fucosylated MMO according to the invention contains only one fucose, i.e. said MMO consists of two or more monosaccharides and only one of said monosaccharides is a fucose.

In a preferred embodiment of the invention, said fucosylated MMO is a neutral oligosaccharide. A “neutral” oligosaccharide as used herein and as generally understood in the state of the art is an oligosaccharide that has no negative charge originating from a carboxylic acid group.

In an additional and/or alternative preferred embodiment, said fucosylated MMO comprises a fucose that is linked to a monosaccharide in an alpha-1,2-, alpha-1,3- or alpha-1,4-linkage, preferably an alpha-1,2- or an alpha-1,3-linkage, more preferably an alpha-1,2-linkage, and wherein said monosaccharide is preferably selected from glucose, N-acetylglucosamine and galactose, more preferably said monosaccharide is glucose or galactose, even more preferably said monosaccharide is galactose. The skilled person will understand that the expression “fucose is linked to a monosaccharide” refers to the situation wherein the fucose is bound to a monosaccharide through a glycosidic bond and wherein said fucose and monosaccharide are part of the oligosaccharide of the invention (which can comprise additional monosaccharide(s) than said fucose and said monosaccharide).

In an additional and/or alternative preferred embodiment, said fucosylated mammalian milk oligosaccharide comprises a lactose, a lacto-N-biose (LNB) or N-acetyllactosamine (LacNAc) at its reducing end, preferably said fucosylated oligosaccharide comprises lactose or LacNAc at is reducing end, more preferably said fucosyalted oligosaccharide comprises lactose at its reducing end.

A fucosylated oligosaccharide comprising lactose at its reducing end is preferably selected form the list consisting of 2′-fucosyllactose (2′FL), 3-fucosyllactose (3-FL), difucosyllactose (diFL), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH), more preferably selected from the list consisting of 2′FL, diFL, LNFP I, GalNAc-LNFP I, Gal-LNFP I, LNDFH I, lewis b-lewis x, DFLNH (a), TFLNH and LNnFP I, even more preferably selected from the list consisting of 2′FL, diFL, LNFP I and LNnFP I, even more preferably wherein said fucosylated mammalian milk oligosaccharide is 2′FL or diFL, most preferably wherein said fucosylated mammalian milk oligosaccharide is 2′FL.

A fucosylated oligosaccharide comprising lacto-N-biose (LNB) at its reducing end is preferably selected form the list consisting of 2′-fucosyllacto-N-biose (2′FLNB), 4-fucosyllacto-N-biose (4FLNB) and difucosyllacto-N-biose (diFLNB), more preferably wherein said fucosylated mammalian milk oligosaccharide is 2′FLNB or diFLNB, most preferably wherein said fucosylated mammalian milk oligosaccharide is 2′FLNB.

A fucosylated oligosaccharide comprising N-acetyllactosamine (LacNac) at its reducing end is preferably selected form the list consisting of 2′-fucosyl-N-acetyllactosamine (2′FlacNAc), difucosyl-N-acetyllactosamine (diFLacNAc) and 3-fucosyl-N-acetyllactosamine (3FlacNAc), more preferably wherein said fucosylated mammalian milk oligosaccharide is 2′FlacNAc or diFLacNAc, most preferably wherein said fucosylated mammalian milk oligosaccharide is 2′FlacNAc.

In a more preferred embodiment, said fucosylated MMO according to the invention is selected from a list consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH). More preferably, said fucosylated MMO according to the invention is selected from the list consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc, even more preferably selected from the list consisting of 2′FL, diFL, LNFP I, LNnFP I, 2′FLNB, diFLNB, 2′FlacNAc and diFLacNAc, even more preferably selected from the list consisting of 2′FL, diFL, 2′FLNB, diFLNB, 2′FlacNAc and diFLacNAc, even more preferably selected from the list consisting of 2′FL, 2′FLNB and 2′FlacNAc, even more preferably wherein said fucosylated mammalian milk oligosaccharide is 2′FL or diFL, most preferably wherein said fucosylated mammalian milk oligosaccharide is 2′FL.

Aforementioned fucosylated mammalian milk oligosaccharides are commercially available and/or the production/purification of these oligosaccharides has been described and hence allows the skilled person to produce/obtain any of said fucosylated oligosaccharides accordingly. For example (each reference is incorporated by reference):
2′FL: Zhou et al, 2021, ACS Synth. Biol. 10 (3): p. 447-458; Lu et al, 2021, ACS Synth. Biol. 10 (5): p. 923-938; WO2022/034073; Carbosynth (OF06739)
3-FL: Zhou et al, 2021; ACS Synth. Biol. 10 (3): p. 447-458, Lu et al, 2021, ACS Synth. Biol. 10 (5): p. 923-938; WO2022/034073; Carbosynth (OF05673)
diFL: WO2022/034073
2′FlacNAc: WO 2022/034075
3FlacNAc: WO 2022/034075
diFLacNAc: WO 2022/034075
2′FLNB: WO 2022/034075
4FLNB: WO 2022/034075
diFLNB: WO 2022/034075
LNFP I: Derya et al, 2020, J. biotech. 319: p. 31-38; Carbosynth (OL05676)
GalNAc-LNFP I: WO 2022/034077
Gal-LNFP I: WO 2022/034077
LNFP II: Zeuner et al, 2018, ACS Synth. Biol. 10 (3): p. 447-458
LNFP III: Zeuner et al, 2018, ACS Synth. Biol. 10 (3): p. 447-458; Carbosynth (OL04212)
LNFP V: WO2020/115671; Carbosynth (OL06817)
LNDFH I: Huang et al, 2021, ACS Catal. 11 (5): p. 2631-2643; Carbosynth (OL01664)
LNDFH II: Yu et al, 2017, chem. Comm. 53 (80): p. 11012-11015; Huang et al, 2021, ACS Catal. 11 (5): p. 2631-2643; Carbosynth (OL06826)
MFLNH III: Carbosynth (OM05898)
DFLNH (a): Carbosynth (OD05375)
DFLNH: Carbosynth (OD06532)
TFLNH: Isosep (57/18-0010)
LNnFP I: Elicityl (GLY033-2-90%)
LNnFP V: Dumon et al, 2001, Glyconj. J. 18 (6): p. 465-474
LNnDFH: Dumon et al, 2001, Glyconj. J. 18 (6): p. 465-474

In an additional and/or alternative more preferred embodiment, said fucosylated MMO according to the invention (and optionally any, preferably all, further mammalian milk oligosaccharides, if also administered to said animal (it is also referred to the Section “Composition”)) has been isolated from a microbial cultivation or fermentation, cell culture, enzymatic reaction or chemical reaction. Alternatively, said fucosylated MMO of the invention (and optionally any, preferably all, further mammalian milk oligosaccharides, if also administered to said animal (it is also referred to the Section “Composition”)) has been isolated by e.g. chromatography or filtration technology from a natural source such as a human or animal milk, preferably animal milk. Isolation of said fucosylated MMO typically results in a solution containing said oligosaccharide. Such a solution can for example be obtained by a method comprising the steps of:
(a) cultivating at least one cell as defined herein, preferably a single cell, that is capable to produce said fucosylated MMO, in a suitable cultivation medium to form a cultivation broth, preferably wherein said cell is metabolically engineered for the production of said fucosylated MMO, and
(b) purifying said fucosylated MMO from the cultivation broth by:
   (i) clarifying the cultivation broth, and
   (ii) removing salts and/or medium components form said clarified cultivation broth, and/or
   (iii) concentrating said fucosylated oligosaccharide in said clarified cultivation broth,
thereby providing a solution comprising a purified oligosaccharide.

In the context of the invention, “clarifying the cultivation broth” refers to the removal of suspended particulates and contaminants, particularly cells, cell components, insoluble metabolites and debris produced by culturing the cell according to the invention. Clarification is preferably one or more of centrifugation, flocculation, decantation and/or filtration.

In the context of the invention, “removing salts and/or medium components form said clarified cultivation broth” refers to removing substantially all the proteins, as well as peptides, amino acids, RNA and DNA and any endotoxins and glycolipids from said clarified cultivation broth, preferably after it has been. In this step, proteins and related impurities can be removed from said saccharide in a conventional manner. Preferably, proteins, salts, by-products, colour, endotoxins and other related impurities are removed from said oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g. using slab-polyacrylamide or sodium dodecyl sulphate-polyacrylamide gel electrophoresis (PAGE)), affinity chromatography (using affinity ligands including e.g. DEAE-Sepharose, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, said oligosaccharide remains in the said oligosaccharide containing clarified cultivation broth. Further purification of said oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the product.

Several drying techniques are known to the skilled person which can be used to obtain powder from a solution containing said oligosaccharide. Powder is preferably obtained by spray drying, freeze drying, spray freeze-drying, crystallization, lyophilization, band or belt drying, drum or roller drying, and/or agitated thin film drying, preferably by spray drying, drum or roller drying, or agitated thin film drying, more preferably by spray drying or agitated thin film drying, most preferably by spray drying, of a solution containing said saccharide. Preferably, said oligosaccharide according to the invention constitutes ≥70.0%, preferably ≥75.0%, more preferably ≥80.0, (w/w) of said powder. Preferably, said powder contains ≤15 wt. %, preferably ≤10 wt. %, more preferably ≤9 wt. %, more preferably ≤8 wt. %, more preferably ≤7 wt. %, even more preferably ≤5 wt. %, even more preferably ≤4 wt. % of liquid, even more preferably ≤3 wt. % of liquid, even more preferably ≤2 wt. % of liquid, most preferably ≤1 wt. %, preferably wherein said liquid is water.

In an additional and/or alternative more preferred embodiment, said fucosylated MMO according to the invention (and optionally any, preferably all, further mammalian milk oligosaccharides, if also administered to said animal (it is also referred to the Section “Composition”)) is provided as a powder in said composition according to the invention. Preferably, said powder is as defined earlier in the present Section.

In an additional and/or alternative more preferred embodiment, said fucosylated MMO according to the invention (and optionally any, preferably all, further mammalian milk oligosaccharides, if also administered to said animal (it is also referred to the Section “Composition”)) has been produced, preferably in vitro and/or ex vivo, by a cell, preferably a single cell, wherein said cell is preferably chosen from the list consisting of a microorganism, a plant cell, an animal cell and a protozoan cell. In other words, said saccharide of the invention has been produced by an in vitro and/or ex vivo culture of cells, wherein said cells are preferably chosen from the list consisting of a microorganism, a plant cell, an animal cell or a protozoan cell.

Preferably, said cell is a microorganism. Preferably, said microorganism is selected from a list consisting of a bacterium, a yeast and a fungus. Further, it is preferred that said cell is genetically engineered for the production of said fucosylated MMO according to the invention. Throughout the description and claims, the terms “has been isolated” and “is obtained” are interchangeably used, unless specifically stated otherwise.

The latter bacterium preferably belongs to the phylum of the Proteobacteria or the phylum of the Firmicutes or the phylum of the Cyanobacteria or the phylum Deinococcus-Thermus, preferably belongs to the phylum of the Proteobacteria. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus, such as Bacillus subtilis or, B. amyloliquefaciens. The latter Bacterium belonging to the phylum Actinobacteria, preferably belonging to the family of the Corynebacteriaceae, with members Corynebacterium glutamicum or C. afermentans, or belonging to the family of the Streptomycetaceae with members Streptomyces griseus or S. fradiae. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g. Saccharomyces cerevisiae, S. bayanus, S. boulardii), Pichia (with members like e.g. Pichia pastoris, P. anomala, P. kluyveri), Komagataella, Hansunella, Kluyveromyces (with members like e.g. Kluyveromyces lactis, K. marxianus, K. thermotolerans), Yarrowia (like e.g. Yarrowia lipolytica), Eremothecium, Zygosaccharomyces, Starmerella (like e.g. Starmerella bombicola) or Debaromyces. The latter yeast is preferably selected from Pichia pastoris, Yarrowia lipolitica, Saccharomyces cerevisiae and Kluyveromyces lactis. The latter fungus belongs preferably to the genus Rhizopus, Dictyostelium, Penicillium, Mucor or Aspergillus. “Plant cells” includes cells of flowering and non-flowering plants, as well as algal cells, for example Chlamydomonas, Chlorella, etc. Preferably, said plant cell is a tobacco, alfalfa, rice, cotton, rapeseed, tomato, corn, maize or soybean cell. The latter animal cell is preferably derived from non-human mammals (e.g. cattle, buffalo, pig, sheep, mouse, rat), birds (e.g. chicken, duck, ostrich, turkey, pheasant), fish (e.g. swordfish, salmon, tuna, sea bass, trout, catfish), invertebrates (e.g. lobster, crab, shrimp, clams, oyster, mussel, sea urchin), reptiles (e.g. snake, alligator, turtle), amphibians (e.g. frogs) or insects (e.g. fly, nematode) or is a genetically modified cell line derived from human cells excluding embryonic stem cells. Both human and non-human mammalian cells are preferably chosen from the list comprising an epithelial cell like e.g. a mammary epithelial cell, mammary myoepithelial cell, mammary progenitor cell, an embryonic kidney cell (e.g. HEK293 or HEK 293T cell), a fibroblast cell, a COS cell, a Chinese hamster ovary (CHO) cell, a murine myeloma cell like e.g. an N20, SP2/0 or YB2/0 cell, an NIH-3T3 cell, a non-mammary adult stem cell or derivatives thereof such as described in WO21067641, preferably mesenchymal stem cell or derivates thereof as described in WO21067641. Said insect cell is preferably derived from Spodoptera frugiperda like e.g. Sf9 or Sf21 cells, Bombyx mori, Mamestra brassicae, Trichoplusia ni like e.g. BTI-TN-5B1-4 cells or Drosophila melanogaster like e.g. Drosophila S2 cells. The latter protozoan cell preferably is a Leishmania tarentolae cell.

In an additional and/or alternative more preferred embodiment, said fucosylated MMO of the invention has been produced, preferably in vitro and/or ex vivo, by a mammary epithelial cell, mammary myoepithelial cell and/or mammary progenitor cell, preferably wherein said cell is generated from non-mammary adult stem cells, more preferably wherein said cell is generated from mesenchymal stem cells. Such cells are well-known to the skilled person, it is in this regard referred to for example WO2021/067641 and WO2021/242866 (mammary epithelial cells derived from non-mammary adult stem cells, preferably from mesenchymal stem cells) and WO2021/142241 (mammary epithelial cells, mammary myoepithelial cells, mammary progenitor cells).

In an even more preferred embodiment, said fucosylated MMO of the invention (and optionally any, preferably all, further mammalian milk oligosaccharides, if also administered to said animal (it is also referred to the Section “Composition”)) has been produced, preferably in vitro and/or ex vivo, by a microorganism cell, preferably said microorganism is a bacterium, a yeast or a fungus, more preferably said microorganism is a bacterium or a yeast, even more preferably said microorganism is a bacterium, most preferably said microorganism is Escherichia coli.

In the context of the invention, it is also within the scope of the present invention that two or more different cells (preferably as defined herein), produce the fucosyalted MMO according to the invention. It is however preferred that a single cell produces said oligosaccharide, i.e. a single culture of said cell produces the oligosaccharide of the invention.

In an additional and/or alternative embodiment, said fucosylated MMO according to the invention (and optionally any, preferably all, further mammalian milk oligosaccharides, if also administered to said animal (it is also referred to the Section “Composition”)) is linked, preferably chemically linked, to a carrier for delivery of said MMO. Preferably, said carrier is a ceramide-based carrier or a polypeptide-based carrier, more preferably said carrier is a ceramide-based carrier. Preferably, said polypeptide-based carrier is epsilon-polylysine, alfa-polylysine, poly (aspartic acid), polyglutamic acid or polyornithine. These carriers are commercially available (e.g. Sigma-Aldrich, Carbosynth). Said ceramide-based carrier is preferably selected from a list consisting of d18: 1/16:0, t18: 0-16:0, t18: 0-h16: 0, t18: 0-h22: 0 and t18: 0-h24: 0. These ceramide carriers are commercially available and well-known to the skilled person and are for example described in WO2010/037785 which is incorporated by reference. d18: 1/16:0 is also known as C16 ceramide and N-palmitoylsphingosine and therefore interchangeable used herein. t18: 0-16:0 is also known as C16 phytoceramide and N-hexadecanoyl phytosphingosine and therefore interchangeable used herein. t18: 0-h16: 0, t18: 0-h22: 0 and t18: 0-h24: 0 are glycosylinositolphosphoceramides (GIPCs). As known to the skilled person, “d” and “t” refer to the hydroxylation state of the whole ceramide or long-chain base moiety (d is 2 groups, t is 3 groups), whereas “h” denotes a hydroxylation of the fatty acyl group.

Said fucosylated mammalian milk oligosaccharide and optionally any, preferably all, further mammalian milk oligosaccharides, if also administered to said animal, is preferably encapsulated (i.e. forming an encapsulate). This is particularly advantageous to protect said oligosaccharide(s) from conditions relating to the manufacturing or storing of a composition according to the invention, especially when said composition is a nutritional composition (it is referred to the Section “Composition”). Such conditions typically entail a high pressure (e.g. a pressure of above 10 bar during extrusion), a high temperature (e.g. a temperature above 100° C. during extrusion and baking), a pH lower than 5, a pH higher than 8, and/or a high moisture content (e.g. wet extrusion). Moreover, other molecules within a composition, especially a nutritional composition, such as proteins could chemically interact with said fucosylated MMO (and any other further MMO if present) especially under the harsh conditions during manufacturing of a nutritional composition. Hence, throughout the application and claims, said “fucosylated MMO” is preferably replaced with “encapsulated fucosylated MMO”. If any further MMO is present (it is also referred to the Section “Composition”), than it is preferred that all MMO's are encapsulated, preferably all MMO's are co-encapsulated.

Said encapsulated is particularly advantageous when the composition according to the invention is a nutritional composition (it is referred to the Section “Composition) which is manufactured by extrusion (dry or wet, preferably wet).

It is preferred that the encapsulate is of the core-shell type, i.e. carrier material forms a shell around the active agent (i.e. the MMO ('s) according to the invention), preferably wherein the core has a median diameter (D (v,0.5)) of 125-250 μm, preferably 150-250 μm, more preferably 165-250 μm, most preferably 165-225 μm, and wherein said encapsulate has a median diameter (D (v,0.5)) which is 20-100 μm, preferably 20-75 μm, more preferably 35-75 μm, longer than said median diameter of the core. The carrier material preferably comprises a hot melt material, more preferably an oil and/or a wax, even more preferably a hydrogenated oil and/or a wax, optionally further comprising starch, preferably wherein said starch constitutes less than 15% (w/w) of said carrier material, more preferably said starch constitutes less than 10% (w/w) of said carrier material.

Said oil is preferably selected from the list consisting of palm oil, sunflower oil, soybean oil, rapeseed oil, coconut oil, babassu oil, palm kernel oil, maize oil, sesame oil and cottonseed oil; more preferably said oil is selected from the list consisting of palm oil, sunflower oil, soybean oil and rapeseed oil; even more preferably said oil is palm oil, most preferably said oil is hydrogenated palm oil.

Said wax is preferably selected from the list consisting of Candelilla wax, Carnauba wax, beeswax, rice bran wax, paraffin wax, jojoba wax, microcrystalline wax and japan wax; more preferably said wax is Candelilla wax or Carnauba wax; most preferably said wax is Candelilla wax.

It is further preferred that the fucosylated MMO or all MMO's present in the encapsulate constitute 5-50% (w/w), preferably 10-40% (w/w), more preferably 20-40% (w/w), even more preferably 20-35% (w/w), of the total weight of the encapsulate.

Throughout the application and claims, it is particularly preferred that said fucosylated MMO according to the invention and optionally any, preferably all, further mammalian milk oligosaccharides, if also administered to said animal, is part of a composition, preferably a composition according to the Section “Composition”.

### Composition

In a preferred embodiment of the first aspect, the invention provides a composition comprising a fucosylated mammalian milk oligosaccharide (MMO), wherein said composition is for use in a method for preventing and/or treating pneumonia in a farmed animal or domesticated animal, preferably a farmed animal.

Said fucosylated MMO is as described in the Section “oligosaccharide”. Said pneumonia is as described in the Section “Pneumonia”. Said method for preventing and/or treating is as described in the Section “Method for preventing and/or treating”. Said animal is as described in the Section “Subject”.

In a preferred embodiment, said fucosylated mammalian milk oligosaccharide (MMO) is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition. Throughout the application and claims, unless specifically stated otherwise, the term “wt. %” means weight by weight (i.e. w/w). If the composition is for example a solid composition, then 1.0 wt. % means 1.0 g fucosylated MMO per 100.0 gram of the solid composition. If the composition is for example a liquid composition, then 1.0 wt. % means 1.0 g fucosylated MMO per 100.0 gram of the liquid composition.

Throughout the application and claims, if said composition is a milk replacer (e.g. calf milk replacer) then the wt. % as recited throughout the application and claims, refers to the drywt. %. If said composition is a (calf) milk replacer, then for example aforementioned paragraph should be read as: “In a preferred embodiment, said fucosylated mammalian milk oligosaccharide (MMO) is present at 0.001-1.000 drywt. %, preferably 0.001-0.750 drywt. %, more preferably 0.001-0.500 drywt. %, even more preferably 0.001-0.400 drywt. %, even more preferably 0.005-0.400 drywt. %, even more preferably 0.005-0.300 drywt. %, even more preferably 0.005-0.250 drywt. %, most preferably 0.005-0.100 drywt. %.” Further, a milk replacer (e.g. calf milk replacer) is often administered to e.g. a calf in a liquid form. Hence, in this context, it is preferred that a liquid milk replacer comprises 10.0-30.0% (w/v), more preferably 12.0-28.0% (w/v), milk replacer powder. Here, it is preferred that the milk replacer powder is constituted in water to obtain said liquid milk replacer. Throughout the application and claims, the term “% w/v)” refers to the weight of the dissolved substance in gram (e.g. milk replacer powder) per 100 milliliters of the final solution (e.g. liquid milk replacer). If said milk replacer is a calf milk replacer, then it is particularly preferred that a liquid calf milk replacer comprises 10.0-20.0% (w/v), more preferably 12.0-20.0% (w/v), even more preferably 12.0-18.0% (w/v), even more preferably 14.0-18.0% (w/v), most preferably 15.0% (w/v), calf milk replacer powder. It is preferred that the calf milk replacer powder is constituted in water to obtain said liquid calf milk replacer. If said milk replacer is a milk replacer for a piglet, then it is particularly preferred that a liquid milk replacer comprises 10.0-30.0% (w/v), more preferably 15.0-30.0% (w/v), even more preferably 20.0-30.0% (w/v), even more preferably 22.0-28.0% (w/v), most preferably 25.0% (w/v), milk replacer powder. It is preferred that the milk replacer powder is constituted in water to obtain said liquid milk replacer.

In a more preferred embodiment, said fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition. This embodiment is for example particularly preferred if said animal is a bovine or a horse, more preferably wherein said animal is a calf or a foal, most preferably wherein said animal is a calf.

In an alternative more preferred embodiment, said fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition. This embodiment is for example particularly preferred if said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.

In a preferred embodiment of the invention, said composition further comprises a second mammalian milk oligosaccharide (MMO), preferably wherein said second mammalian milk oligosaccharide is a sialylated MMO, more preferably wherein said second mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.

Preferably, said second MMO consists of 3-8, more preferably 3-7, even more preferably 3-6, most preferably 3-5, monosaccharides.

A sialylated MMO comprising lactose at its reducing end is preferably selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c and LST d; more preferably selected from the list consisting of 3′SL, 6′SL, 3,6-disialyllactose, 6,6′-disialyllactose, 8,3-disialyllactose, LST a, LST b, LST c and LST d; even more preferably selected from the list consisting of 3′SL, 6′SL, LST a, LST b, LST c and LST d; most preferably wherein said sialylated MMO is 3′-sialyllactose (3′SL) or 6′-sialyllactose (6′SL).

A sialylated MMO comprising lacto-N-biose (LNB) at its reducing end is preferably selected form the list consisting of 3′-sialyllacto-N-biose (3′SLNB), 6′-sialyllacto-N-biose (6′SLNB) and monofucosylmonosialyllacto-N-octaose (sialyl Lewis a); more preferably said sialylated MMO is 3′SLNB or 6′SLNB.

A sialylated MMO comprising N-acetyllactosamine (LacNac) at its reducing end is preferably selected form the list consisting of 3′-sialyllactosamine (3′SLacNAc), 6′-sialyllactosamine (6′SLacNAc) and sialyl Lewis x; more preferably said sialylated MMO is 3′SLacNAc or 6′SLacNAc.

When a second MMO, preferably a sialylated MMO, is present in said composition, then it is a preferred embodiment that the amount of said second MMO in said composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO in said composition. It is a more preferred embodiment that the amount of said fucosylated MMO in said composition is lower than the amount of said second MMO, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said second MMO in said composition. It is an even more preferred embodiment that, when said second MMO is a sialylated MMO, that the amount of said fucosylated MMO in said composition is lower than the amount of said second MMO, preferably said amount of said fucosylated MMO is 1.1 to 10.0, preferably 1.5-7.5, more preferably 2.5-7.5, most preferably 4.0-7.0, times lower than the amount of said second MMO in said composition.

In an additional preferred embodiment, said composition further comprises a third mammalian milk oligosaccharide (MMO), preferably wherein said third mammalian milk oligosaccharide is a sialylated MMO, more preferably wherein said third mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.

Preferably, said third MMO consists of 3-8, more preferably 3-7, even more preferably 3-6, most preferably 3-5, monosaccharides.

A sialylated MMO comprising lactose at its reducing end is preferably selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c and LST d; more preferably selected from the list consisting of 3′SL, 6′SL, 3,6-disialyllactose, 6,6′-disialyllactose, 8,3-disialyllactose, LST a, LST b, LST c and LST d; even more preferably selected from the list consisting of 3′SL, 6′SL, LST a, LST b, LST c and LST d; most preferably wherein said sialylated MMO is 6′-sialyllactose (6′SL) or 3′-sialyllactose (3′SL) or.

A sialylated MMO comprising lacto-N-biose (LNB) at its reducing end is preferably selected form the list consisting of 3′-sialyllacto-N-biose (3′SLNB), 6′-sialyllacto-N-biose (6′SLNB) and monofucosylmonosialyllacto-N-octaose (sialyl Lewis a); more preferably said sialylated MMO is 3′SLNB or 6′SLNB.

A sialylated MMO comprising N-acetyllactosamine (LacNac) at its reducing end is preferably selected form the list consisting of 3′-sialyllactosamine (3′SLacNAc), 6′-sialyllactosamine (6′SLacNAc) and sialyl Lewis x; more preferably said sialylated MMO is 3′SLacNAc or 6′SLacNAc.

When a third MMO, preferably a sialylated MMO, is present in said composition, then it is a preferred embodiment that the amount of said third MMO in said composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO in said composition. It is a more preferred embodiment that the amount of said fucosylated MMO in said composition is lower than the amount of said third MMO, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said third MMO in said composition. It is an even more preferred embodiment that, when said third MMO is a sialylated MMO, that the amount of said fucosylated MMO in said composition is lower than the amount of said third MMO, preferably said amount of said fucosylated MMO is 1.1 to 10.0, preferably 1.5-7.5, more preferably 1.5-5.0, most preferably 2.5-5.0, times lower than the amount of said third MMO in said composition.

When a second MMO (preferably a sialylated MMO, i.e. a first sialylated MMO) and a third MMO (preferably a sialylated MMO, i.e. a second sialylated MMO) are present in said composition, then it is a preferred embodiment that the ratio between the amount of said second MMO and the amount of said third MMO in said composition is similar to this ratio as found in the mother's milk of the same species. Throughout the application and claims, unless specifically stated otherwise, a “similar” ratio preferably refers to a ratio which is identical or falls in the range of ratio+/−20% of said ratio, preferably ratio+/−10% of said ratio. When the animal is a bovine, the second MMO is a sialylated MMO and the third MMO is a sialylated MMO, said ratio is preferably 1.1 to 6.0, more preferably 1.1 to 5.0, even more preferably 1.25 to 4.0, even more preferably 1.25 to 3.0, even more preferably 1.5 to 3.0, most preferably 1.5 to 2.5. When the animal is a horse, the second MMO is a sialylated MMO and the third MMO is a sialylated MMO, said ratio is preferably 1.1 to 15.0, more preferably 1.1 to 12.5, even more preferably 1.1 to 10.0, even more preferably 1.5 to 10.0, even more preferably 2.5 to 10, even more preferably 5.0 to 10.0, even more preferably 7.5 to 10.0, most preferably 8.0 to 10.0. When the animal is a pig, the second MMO is a sialylated MMO and the third MMO is a sialylated MMO, said ratio is preferably 7.5-40.0, more preferably 7.5-35.0, even more preferably 10.0-35.0, even more preferably 15.0-35.0, even more preferably 20.0-35.0, most preferably 25.0-35.0. When the animal is a goat, the second MMO is a sialylated MMO and the third MMO is a sialylated MMO, said ratio is preferably 0.25-5.0, more preferably 0.25-3.0, even more preferably 0.25-2.0, even more preferably 0.25-1.5, most preferably 0.5-1.5. Further, it is a more preferred embodiment that the amount of said second MMO in said composition is higher than the amount of said third MMO, preferably said amount of said second MMO is 1.1 to 40.0, more preferably 1.1 to 35.0, most preferably 1.5 to 35.0, times the amount of said third MMO in said composition. Further, it is an even more preferred embodiment that the amount of said second MMO, when said second MMO is a sialylated MMO, in said composition is higher than the amount of said third MMO, when said third MMO is a sialylated MMO, preferably said amount of said second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of said third MMO in said composition.

In a more preferred embodiment, said composition further comprises a second mammalian milk oligosaccharide (MMO), which is a sialylated MMO selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. More preferably said second MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said second MMO is selected from the list consisting of 3′SL, 6′SL, 3′SLNB, 6′SLNB, 3′SLacNAc and 6′SLacNAc. Most preferably said second MMO is 3′-sialyllactose (3′SL) or 6′-sialyllactose (6′SL).

In an additional more preferred embodiment, said composition further comprises a third mammalian milk oligosaccharide (MMO), which is a sialylated MMO selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x. More preferably said third MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x. Even more preferably said third MMO is selected from the list consisting of 3′SL, 6′SL, 3′SLNB, 6′SLNB, 3′SLacNAc and 6′SLacNAc. Most preferably said third MMO is 6′-sialyllactose (6′SL) or 3′-sialyllactose (3′SL).

In the context of the present invention, said second mammalian milk oligosaccharide and said third mammalian milk oligosaccharide preferably do not contain N-acetyllactosamine.

The inventors have surprisingly found that a fucosylated MMO according to the invention, preferably a composition comprising a fucosylated MMO according to the invention, is sufficient to obtain an effective therapeutic for preventing and/or treating pneumonia as described herein. The presence of other (oligo) saccharides is not required to obtain said therapeutic effect. While the composition according to the invention can include additional (oligo) saccharides, the present invention relates to the fact that only the fucosylated MMO according to the invention is required for achieving an efficient therapeutic effect as described herein.

In this context, it is hence a preferred embodiment that said composition according to the invention does not comprise lacto-N-tetraose (LNT).

In an additional and/or alternative preferred embodiment, said composition according to the invention does not comprise lacto-N-neotetraose (LNnT).

In an additional and/or alternative preferred embodiment, said composition according to the invention does not comprise N-acetyllactosamine.

In an additional and/or alternative preferred embodiment, said composition according to the invention does not comprise an oligosaccharide containing N-acetyllactosamine, preferably does not comprise a saccharide containing N-acetyllactosamine.

In an additional and/or alternative preferred embodiment, said composition according to the invention does not comprise 3′sialyllactose.

In an additional and/or alternative preferred embodiment, said composition according to the invention does not comprise 6′sialyllactose.

More preferably, said composition according to the invention does not comprise a sialylated mammalian milk oligosaccharide, even more preferably does not comprise a sialylated oligosaccharide, most preferably does not comprise a sialylated saccharide.

In an additional and/or alternative preferred embodiment, said composition according to the invention does not comprise a fructo-oligosaccharide (FOS).

In an additional and/or alternative preferred embodiment, said composition according to the invention does not comprise a galactose-oligosaccharide (GOS).

In an additional and/or alternative preferred embodiment, said composition according to the invention does not comprise an isomaltooligosaccharide.

In a more preferred embodiment, said composition according to the invention comprises said fucosylated MMO as described herein, optionally a second MMO as described herein and optionally a third MMO as described herein, and no additional MMOs (preferably no additional oligosaccharides, more preferably no additional saccharides).

In an even more preferred embodiment, said composition according to the invention comprises said fucosylated MMO as described herein, and no additional MMOs (preferably no additional oligosaccharides, more preferably no additional saccharides).

In an even more preferred embodiment, said composition according to the invention does not comprise a neutral non-fucosylated MMO.

In an even more preferred embodiment, said composition according to the invention comprises only one mammalian milk oligosaccharide, i.e. said fucosylated MMO as described herein.

In a preferred embodiment, said composition according to the invention is not mother's milk.

In an additional and/or alternative preferred embodiment, said composition according to the invention is a synthetic composition.

In the context of the present invention, a “synthetic composition” or a “synthetic nutritional composition” refers to a composition which is artificially prepared and preferably refers to a composition comprising at least one component that is produced ex vivo, either chemically and/or biologically, e.g. by means of chemical reaction, enzymatic reaction or recombinantly, or purified by humans. It is preferred that a synthetic composition of the invention is not identical with a naturally occurring composition.

In this context, it is preferred that the fucosylated MMO as described herein has been isolated from a microbial cultivation or fermentation, cell culture, enzymatic reaction or chemical reaction. Or has been isolated from a natural source (it is referred to the Section “Oligosaccharide”). It is more preferred that the fucosylated MMO as described herein has been produced in vitro and/or ex vivo by a cell (it is referred to the Section “Oligosaccharide”).

In an additional and/or alternative preferred embodiment, said composition according to the invention further comprises a microbial strain which does not use, preferably is not able to use, said fucosylated MMO as a carbon source.

In an alternative preferred embodiment, said composition according to the invention does not comprise a microbial strain. Examples of such a strain is Bifidobacterium, Lactobacillus and Saccharomyces boulardii.

In a preferred embodiment, said composition according to the invention is a pharmaceutical composition, optionally further comprising a pharmaceutically acceptable carrier, filler, preservative, solubilizer, diluent, excipient, salt, adjuvant and/or solvent.

In an additional and/or alternative preferred embodiment, said composition is a nutritional composition, optionally further comprising a feed ingredient and/or a food ingredient, wherein said feed/food ingredient is preferably chosen from the list consisting of:
a lipid, preferably one or more selected from the list consisting of an oil, fat, ester, monoglyceride, diglyceride, triglyceride and free fatty acid;
a vitamin, preferably one or more selected from the list consisting of vitamin A, vitamin B, vitamin C, vitamin D, vitamin E and vitamin H, or a derivate thereof;
an amino acid compound;
a trace element;
a mineral, preferably one or more selected from the list consisting of calcium, phosphorus, magnesium, iron, zinc, manganese, copper, sodium, potassium, molybdenum, chromium, selenium and chloride;
an antioxidant;
a prebiotic agent, preferably one or more selected from the list consisting of GOS (galacto-oligosaccharide), FOS (fructo-oligosaccharide), inulin and resistant starch;
a carbohydrate;
an antimicrobial agent; and/or
a protein.

Generally, any source of protein may be used so long as it is suitable for nutritional compositions and is otherwise compatible with any other selected ingredients or features in the nutritional composition. Non-limiting examples of suitable proteins (and sources thereof) suitable for use in the nutritional composition according to the invention include, but are not limited to, intact, hydrolyzed, or partially hydrolyzed protein, which may be derived from any known or otherwise suitable source such as milk (e.g., casein, whey), animal (e.g., meat, fish), cereal (e.g., rice, corn, wheat), vegetable (e.g., soy, pea, potato, bean), and combinations thereof. The protein may also include a mixture of amino acids (often described as free amino acids) known for use in nutritional products or a combination of such amino acids with the intact, hydrolyzed, or partially hydrolyzed proteins described herein. The amino acids may be naturally occurring or synthetic amino acids. More particular examples of suitable protein (or sources thereof) used in a nutritional composition according to the invention include, but are not limited to, whole cow's milk, partially or completely defatted milk, milk protein concentrates, milk protein isolates, nonfat dry milk, condensed skim milk, whey protein concentrates, whey protein isolates, acid caseins, sodium caseinates, calcium caseinates, potassium caseinates, legume protein, soy protein concentrates, soy protein isolates, pea protein concentrates, pea protein isolates, collagen proteins, potato proteins, rice proteins, wheat proteins, canola proteins, quinoa, insect proteins, earthworm proteins, fungal (e.g., mushroom) proteins, hydrolyzed yeast, gelatin, bovine colostrum, human colostrum, glycol macropeptides, mycoproteins, proteins expressed by microorganisms (e.g., bacteria and algae), and combinations thereof. A nutritional composition according to the invention may include any individual source of protein or combination of the various sources of protein listed above. In addition, the proteins for use herein can also include, or be entirely or partially replaced by, free amino acids known for use in nutritional products, non-limiting examples of which include L-tryptophan, L-glutamine, L-tyrosine, L-methionine, L-cysteine, taurine, L-arginine, L-carnitine, and combinations thereof.

The carbohydrate or source of carbohydrate suitable for use in a nutritional composition according to the invention may be simple, complex, or variations or combinations thereof. Generally, the carbohydrate may include any carbohydrate or carbohydrate source that is suitable for use nutritional compositions and is otherwise compatible with any other selected ingredients or features in the nutritional composition. Non-limiting examples of carbohydrates suitable for use in the nutritional composition according to the invention, but are not limited to, polydextrose, maltodextrin; hydrolyzed or modified starch or cornstarch; glucose polymers; corn syrup; corn syrup solids; sucrose; glucose; fructose; lactose; high fructose corn syrup; honey; sugar alcohols (e.g., maltitol, erythritol, sorbitol); isomaltulose; sucromalt; pullulan; potato starch; and other slowly-digested carbohydrates; dietary fibers including, but not limited to, fructooligosaccharides (FOS), galactooligosaccharides (GOS), oat fiber, soy fiber, gum arabic, sodium carboxymethylcellulose, methylcellulose, guar gum, gellan gum, locust bean gum, konjac flour, hydroxypropyl methylcellulose, tragacanth gum, karaya gum, gum acacia, chitosan, arabinogalactans, glucomannan, xanthan gum, alginate, pectin, low methoxy pectin, high methoxy pectin, cereal beta-glucans (e.g., oat beta-glucan, barley beta-glucan), carrageenan and psyllium, soluble and insoluble fibers derived from fruits or vegetables; other resistant starches; and combinations thereof. A nutritional composition according to the invention may include any individual source of carbohydrate or combination of the various sources of carbohydrate listed above.

The fat or source of fat suitable for use in a nutritional composition according to the invention may be derived from various sources including, but not limited to, plants, animals, and combinations thereof. Generally, the fat may include any fat or fat source that is suitable for use in a nutritional composition according to the invention and is otherwise compatible with any other selected ingredients or features in the nutritional composition. Non-limiting examples of suitable fat (or sources thereof) for use in a nutritional composition according to the invention include coconut oil, fractionated coconut oil, soy oil, high oleic soy oil, corn oil, olive oil, safflower oil, high oleic safflower oil, medium chain triglyceride oil (MCT oil), high gamma linolenic (GLA) safflower oil, sunflower oil, high oleic sunflower oil, palm oil, palm kernel oil, palm olein, canola oil, high oleic canola oil, marine oils, fish oils, algal oils, borage oil, cottonseed oil, fungal oils, eicosapentaenoic acid (EPA), docosahexaenoic acid (DHA), arachidonic acid (ARA), conjugated linoleic acid (CLA), alpha-linolenic acid, rice bran oil, wheat bran oil, interesterified oils, transesterified oils, structured lipids, and combinations thereof. Generally, the fats used in a nutritional composition for formulating infant formulas and paediatric formulas provide fatty acids needed both as an energy source and for the healthy development of the infant, toddler, or child. These fats typically comprise triglycerides, although the fats may also comprise diglycerides, monoglycerides, and free fatty acids. Fatty acids provided by the fats in the nutritional composition include, but are not limited to, capric acid, lauric acid, myristic acid, palmitic acid, palmitoleic acid, stearic acid, oleic acid, linoleic acid, alpha-linolenic acid, ARA, EPA, and DHA. The nutritional composition can include any individual source of fat or combination of the various sources of fat listed above. Preferably, the fat is a mixture of vegetable fat and milk fat such as obtained from milk from a mammal like cow, sheep, goat, mare, or camel. More preferably, wherein the milk fat is bovine milk fat. Mixtures of different types of fat are preferred because they help to provide different fatty acids and better resemble the type of linkage between the glycerol moiety and the fatty acid moiety in the fat, when compared to human mother's milk.

When one or more prebiotics is/are present, then it is preferred that the weight ratio between the oligosaccharide according to the invention and said prebiotic(s) is in the range of from 0.5:10 to 10:0.5.

Preferably, said pharmaceutical or nutritional composition according to the invention comprises one or more probiotics for its beneficial effect on the subject's gut microbiome. Examples of probiotics include Bifidobacterium, Lactobacillus and Saccharomyces boulardii.

In a more preferred embodiment, said nutritional composition is a synthetic nutritional composition.

In an additional and/or alternative more preferred embodiment, said nutritional composition is selected from a list consisting of a milk replacer, creep feed, prestarter diet, starter diet, pre-weaning feed, weaning feed and post-weaning feed, preferably selected form the list consisting of a milk replacer, starter diet, creep feed and post-weaning feed, more preferably said nutritional composition is a milk replacer or starter diet, most preferably a milk replacer.

In the context of the invention, the term “creep feed” refers to a nutritional composition which is used to supplement a pre-weaned animal with a solid diet while said animal is suckling. For example, a piglet receives creep feed to ease the transition from sow's milk to solid pig starter feed. The creep feed stimulates the digestive system of the piglet to produce e.g. amylase which digests carbohydrates in dry feed. If a pig is better able to digest dry feed, he can start eating quickly post-weaning for better performance.

In the context of the invention, the term “milk replacer” refers to a nutritional composition which serves as a substitute for mother's milk. A milk replacer comprises milk proteins, fat, carbohydrates, vitamins and minerals. Preferably, a milk replacer comprises 10 drywt. % to 28 drywt. % of at least one protein, 13 drywt. % to 28 drywt. % (preferably 20 drywt. % to 25 drywt. %) of at least one fat and lactose at <55 drywt. %. More preferably, a milk replacer comprises 18 drywt. % to 24 drywt. % of at least one protein, 15 drywt. % to 28 drywt. % (preferably 20 drywt. % to 25 drywt. %) of at least one fat and lactose at <50 drywt. %. In this context, “drywt. %” refers to the ratio between the weight of the recited component (e.g. 2′FL) and the dry weight of the milk replacer (w/v %). Throughout the application and claims, a milk replacer is preferably a calf milk replacer.

Method for Preventing and/or Treating Pneumonia

In a second aspect, the invention provides a method for preventing and/or treating pneumonia in an animal, wherein said animal is a farmed animal or a domesticated animal, preferably a farmed animal. The method comprises administering a fucosylated mammalian milk oligosaccharide (MMO), preferably a composition comprising a fucosylated mammalian milk oligosaccharide (MMO). Said fucosylated MMO and said composition are as described in the first aspect of the invention.

In the context of the present invention, said “method for preventing and/or treating”, “pneumonia”, “animal”, “composition”, and “fucosylated mammalian milk oligosaccharide” as described throughout the second aspect of the present invention and claims are as described in the first aspect of the invention.

### Use

In a third aspect, the invention provides the use of a fucosylated mammalian milk oligosaccharide (MMO), preferably a composition comprising a fucosylated mammalian milk oligosaccharide (MMO) (composition as described in the first aspect of the invention), for the manufacture of a medicament for preventing and/or treating pneumonia in an animal, wherein said animal is a farmed animal or a domesticated animal, preferably a farmed animal.

In the context of the present invention, said “method for preventing and/or treating”, “pneumonia”, “animal”, “composition”, and “fucosylated mammalian milk oligosaccharide” as described throughout the third aspect of the present invention and claims are as described in the first aspect of the invention.

### SPECIFIC EMBODIMENTS

The present invention preferably relates to the following specific embodiments:
1. A fucosylated mammalian milk oligosaccharide (MMO) for use in a method for preventing and/or treating pneumonia in farmed or domesticated animal, preferably a farmed animal.
2. A fucosylated MMO for use according to embodiment 1, wherein said pneumonia is acute pneumonia.
3. A fucosylated MMO for use according to embodiment 1 or 2, wherein said pneumonia is shipping fever pneumonia.
4. A fucosylated MMO for use according to embodiment 1 or 2, wherein said pneumonia is brooder pneumonia.
5. A fucosylated MMO for use according to any one of embodiments 1 to 4, wherein said farmed animal or domesticated animal is selected from the list consisting of bovine, pig, sheep, goat, horse, camelid (preferably llama or alpaca), dog, cat, rabbit and poultry; more preferably selected from the list consisting of bovine, pig, sheep, goat, horse, camelid (preferably llama or alpaca) and poultry; even more preferably selected from the list consisting of bovine, sheep, goat, horse, camelid (preferably llama or alpaca) and poultry; even more preferably selected from the list consisting of bovine, horse and poultry; most preferably said animal is bovine.
6. A fucosylated MMO for use according to any one of embodiments 1 to 5, wherein said animal is a non-adult animal.
7. A fucosylated MMO for use according to embodiment 6, wherein said farmed animal or domesticated animal is selected from the list consisting of a calf, piglet, lamb, kid, foal, cria, puppy, kitten, kit and a chick; more preferably selected from the list consisting of calf, piglet, lamb, kid, foal, cria and chick; even more preferably selected from the list consisting of a calf, lamb, kid, foal, cria and a chick; even more preferably selected from the list consisting of a calf, foal and chick; most preferably said animal is a calf.
8. A fucosylated MMO for use according to any one of embodiments 1 to 7, wherein said fucosylated MMO consists of 3-8, preferably 3-7, more preferably 3-6, even more preferably 3-5, monosaccharides.
9. A fucosylated MMO for use according to any one of embodiments 1 to 8, wherein said fucosylated MMO is a neutral oligosaccharide.
10. A fucosylated MMO for use according to any one of embodiments 1 to 9, wherein said fucosylated MMO comprises a fucose that is linked to a monosaccharide in an alpha-1,2-, alpha-1,3- or alpha-1,4-linkage, preferably an alpha-1,2- or an alpha-1,3-linkage, more preferably an alpha-1,2-linkage, and wherein said monosaccharide is preferably selected from glucose, N-acetylglucosamine and galactose, more preferably said monosaccharide is glucose or galactose, even more preferably said monosaccharide is galactose.
11. A fucosylated MMO for use according to any one of embodiments 1 to 10, wherein said fucosylated MMO comprises a lactose, a lacto-N-biose (LNB) or N-acetyllactosamine (LacNAc) at its reducing end, preferably said fucosylated oligosaccharide comprises lactose or LacNAc at is reducing end, more preferably said fucosyalted oligosaccharide comprises lactose at its reducing end.
12. A fucosylated MMO for use according to any one of embodiments 1 to 11, wherein said fucosylated MMO is selected from the list consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH); more preferably selected from the list consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc; even more preferably selected from the list consisting of 2′FL, diFL, LNFP I, LNnFP I, 2′FLNB, diFLNB, 2′FlacNAc and diFLacNAc; even more preferably selected from the list consisting of 2′FL, diFL, 2′FLNB, diFLNB, 2′FlacNAc and diFLacNAc; even more preferably selected from the list consisting of 2′FL, 2′FLNB and 2′FlacNAc; even more preferably wherein said fucosylated MMO is 2′FL or diFL, most preferably wherein said fucosylated MMO is 2′FL.
13. A fucosylated MMO for use according to any one of embodiments 1 to 12, wherein said fucosylated MMO is selected from the list consisting of 2′FL, diFL, LNFP I, LNnFP I, 2′FLNB, diFLNB, 2′FlacNAc and diFLacNAc.
14. A fucosylated MMO for use according to any one of embodiments 1 to 13, wherein said fucosylated MMO is 2′-fucosyllactose (2′FL).
15. A fucosylated MMO for use according to any one of embodiments 1 to 14, wherein said fucosylated MMO is part of a composition.
16. A fucosylated MMO for use according to embodiment 15, wherein said fucosylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
17. A fucosylated MMO for use according to embodiment 15 or 16, wherein said fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition; preferably wherein said animal is a bovine or a horse, more preferably a calf or a foal, even more preferably a calf.
18. A fucosylated MMO for use according to embodiment 15 or 16, wherein said fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
19. A fucosylated MMO for use according to any one of embodiments 1 to 18, wherein said method comprises administering an effective amount of said fucosylated MMO.
20. A fucosylated MMO for use according to any one of embodiments 1 to 19, wherein said fucosylated MMO is administered to said animal at least once, preferably at least twice, between birth (0 weeks) and 5 weeks of age, preferably between birth and 4 weeks of age, more preferably between birth and 3 weeks of age.
21. A fucosylated MMO for use according to any one of embodiments 1 to 20, wherein said fucosylated MMO is administered to said animal at least once, preferably at least twice, more preferably daily:
   in the three weeks, preferably two weeks, more preferably one week, preceding the day of transporting said animal; and/or
   on the day of transporting said animal; and/or
   in the three weeks, preferably two weeks, more preferably one week, after the day of transporting said animal.
22. A fucosylated MMO for use according to any one of embodiments 1 to 21, wherein said fucosylated MMO is administered to said animal for at least 1 week, preferably at least 2 weeks, more preferably at least 3 weeks, even more preferably at least 4 weeks, most preferably at least 6 weeks.
23. A fucosylated MMO for use according to any one of embodiments 1 to 22, wherein said fucosylated MMO is administered to said animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.
24. A fucosylated MMO for use according to any one of embodiments 1 to 23, wherein said fucosylated MMO is administered to said animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.
25. A fucosylated MMO for use according to any one of embodiments 1 to 24, wherein said fucosylated MMO is administered to said animal for:
   if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
   if subject is a pig: 1-18, preferably 1-12, more preferably 1-8, even more preferably 1-6, even more preferably 1-4, most preferably 2-4, consecutive weeks;
   if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a horse: 1-18, preferably 1-12, more preferably 1-8, even more preferably 1-6, even more preferably 1-4, most preferably 2-4, consecutive weeks;
   if subject is a camelid: 1-18, preferably 1-12, more preferably 1-8, even more preferably 1-6, even more preferably 1-4, most preferably 2-4, consecutive weeks;
   if subject is a rabbit: 1-12, preferably 1-8, more preferably 1-6, even more preferably 1-4, most preferably 2-4, consecutive weeks;
   if subject is a dog: 1-12, preferably 1-8, more preferably 1-6, even more preferably 1-4, most preferably 2-4, consecutive weeks;
   if subject is a cat: 1-12, preferably 1-8, more preferably 1-6, even more preferably 1-4, most preferably 2-4, consecutive weeks;
   if subject is poultry: 1-8, preferably 1-6, more preferably 1-4, most preferably 2-4, consecutive weeks.
26. A fucosylated MMO for use according to any one of embodiments 1 to 25, wherein said fucosylated MMO is administered at least once a week, preferably at least once every 3 days, more preferably at least once every 2 days, most preferably at least once daily.
27. A fucosylated MMO for use according to any one of embodiments 1 to 26, wherein said fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
28. A fucosylated MMO for use according to any one of embodiments 1 to 26, wherein said fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
29. A fucosylated MMO for use according to any one of embodiments 1 to 26, wherein said fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein said animal is a bovine or a horse, more preferably a calf or a foal, even more preferably a calf.
30. A fucosylated MMO for use according to any one of embodiments 1 to 26, wherein said fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
31. A fucosylated MMO for use according to any one of embodiments 1 to 30, wherein said fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of said animal.
32. A fucosylated MMO for use according to any one of embodiments 1 to 30, wherein said fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of said animal; preferably wherein said animal is a bovine or a horse, more preferably a calf or a foal, even more preferably a calf.
33. A fucosylated MMO for use according to any one of embodiments 1 to 30, wherein said fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of said animal; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
34. A fucosylated MMO for use according to any one of embodiments 1 to 33, wherein said method further comprises administering a second mammalian milk oligosaccharide (MMO), preferably wherein said second mammalian milk oligosaccharide is a sialylated MMO, more preferably wherein said second mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.
35. A fucosylated MMO for use according to embodiment 34, wherein said second MMO is selected from the list consisting of 3′-sialyllactose (3′SL), 6′-sialyllactose (6′SL), 3,6-disialyllactose, 6,6′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably said second MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably said second MMO is selected from the list consisting of 3′SL, 6′SL, 3′SLNB, 6′SLNB, 3′SLacNAc and 6′SLacNAc; most preferably said second MMO is 3′-sialyllactose (3′SL) or 6′-sialyllactose (6′SL).
36. A fucosylated MMO for use according to embodiment 34 or 35, wherein said second MMO is 3′-sialyllactose (3′SL) or 6′-sialyllactose (6′SL).
37. A fucosylated MMO for use according to any one of embodiments 34 to 36, wherein said fucosylated MMO and said second MMO are part of a composition.
38. A fucosylated MMO for use according to any one of embodiments 34 to 37, wherein the amount of said fucosylated MMO administered to said animal is lower than the amount of said second MMO administered to said animal, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said second MMO administered to said animal.
39. A fucosylated MMO for use according to any one of embodiments 34 to 37, wherein the amount of said fucosylated MMO administered to said animal is lower than the amount of said second MMO administered to said animal, preferably said amount of said fucosylated MMO is 1.1 to 10.0, preferably 1.5-7.5, more preferably 2.5-7.5, most preferably 4.0-7.0, times lower than the amount of said second MMO administered to said animal.
40. A fucosylated MMO for use according to any one of embodiments 34 to 37, wherein the amount of said second MMO administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal.
41. A fucosylated MMO for use according to any one of embodiments 1 to 40, wherein said method further comprises administering a third mammalian milk oligosaccharide (MMO), preferably wherein said third mammalian milk oligosaccharide is a sialylated MMO, more preferably wherein said third mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.
42. A fucosylated MMO for use according to embodiment 41, wherein said third MMO is selected from the list consisting of 3′-sialyllactose (3′SL), 6′-sialyllactose (6′SL), 3,6-disialyllactose, 6,6′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably said third MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably said third MMO is selected from the list consisting of 3′SL, 6′SL, 3′SLNB, 6′SLNB, 3′SLacNAc and 6′SLacNAc; most preferably said third MMO is 6′-sialyllactose (6′SL) or 3′-sialyllactose (3′SL).
43. A fucosylated MMO for use according to embodiment 41 or 42, wherein said third MMO is 6′-sialyllactose (6′SL) or 3′-sialyllactose (3′SL).
44. A fucosylated MMO for use according to any one of embodiments 41 to 43, wherein said fucosylated MMO, said second MMO and said third MMO are part of a composition.
45. A fucosylated MMO for use according to any one of embodiments 41 to 44, wherein the amount of said fucosylated MMO administered to said animal is lower than the amount of said third MMO administered to said animal, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said third MMO administered to said animal.
46. A fucosylated MMO for use according to any one of embodiments 41 to 44, wherein the amount of said fucosylated MMO administered to said animal is lower than the amount of said third MMO administered to said animal, preferably said amount of said fucosylated MMO is 1.1 to 10.0, preferably 1.5-7.5, more preferably 1.5-5.0, most preferably 2.5-5.0, times lower than the amount of said third MMO administered to said animal.
47. A fucosylated MMO for use according to any one of embodiments 41 to 44, wherein the amount of said third MMO administered to said animal is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO administered to said animal.
48. A fucosylated MMO for use according to any one of embodiments 41 to 47, wherein the ratio between the amount of said second MMO and the amount of said third MMO administered to said animal is similar to this ratio as found in the mother's milk of the same species.
49. A fucosylated MMO for use according to any one of embodiments 41 to 48, wherein the amount of said second MMO administered to said animal is higher than the amount of said third MMO administered to said animal, preferably said amount of said second MMO is 1.1 to 40.0, more preferably 1.1 to 35.0, most preferably 1.5 to 35.0, times the amount of said third MMO administered to said animal.
50. A fucosylated MMO for use according to any one of embodiments 41 to 48, wherein the amount of said second MMO administered to said animal is higher than the amount of said third MMO administered to said animal, preferably said amount of said second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of said third MMO administered to said animal.
51. A fucosylated MMO for use according to any one of embodiments 1 to 50, wherein said method does not comprise administering lacto-N-tetraose (LNT).
52. A fucosylated MMO for use according to any one of embodiments 1 to 51, wherein said method does not comprise administering lacto-N-neotetraose (LNnT).
53. A fucosylated MMO for use according to any one of embodiments 1 to 52, wherein said method does not comprise administering N-acetyllactosamine.
54. A fucosylated MMO for use according to any one of embodiments 1 to 53, wherein said method does not comprise administering an oligosaccharide containing N-acetyllactosamine, preferably does not comprise administering a saccharide containing N-acetyllactosamine.
55. A fucosylated MMO for use according to any one of embodiments 1 to 54, wherein said method does not comprise administering a fructo-oligosaccharide (FOS).
56. A fucosylated MMO for use according to any one of embodiments 1 to 55, wherein said method does not comprise administering a galacto-oligosaccharide (GOS).
57. A fucosylated MMO for use according to any one of embodiments 1 to 56, wherein said method does not comprise administering an isomaltooligosaccharide.
58. A fucosylated MMO for use according to any one of embodiments 1 to 57, wherein said method does not comprise administering a neutral non-fucosylated MMO.
59. A fucosylated MMO for use according to any one of embodiments 1 to 33 and 51 to 58, wherein said method does not comprise administering a further mammalian milk oligosaccharide.
60. A fucosylated MMO for use according to any one of embodiments 1 to 59, wherein said method further comprises administering a microbial strain which does not use, preferably is not capable to use, said fucosylated MMO as carbon source.
61. A fucosylated MMO for use according to any one of embodiments 1 to 59, wherein said method does not comprise administering a microbial strain.
62. A fucosylated MMO composition for use according to any one of embodiments 15 to 18, 37 and 44, wherein said composition is not mother's milk.
63. A fucosylated MMO for use according to any one of embodiments 15 to 18, 37, 44 and 62, wherein said composition is a synthetic composition.
64. A fucosylated MMO for use according to any one of embodiments 1 to 63, wherein said fucosylated MMO has been produced in vitro and/or ex vivo by a cell, preferably a single cell, wherein said cell is preferably chosen from the list consisting of a microorganism, a plant cell, an animal cell and a protozoan cell.
65. A fucosylated MMO for use according to any one of embodiments 1 to 64, wherein said fucosylated MMO is linked, preferably chemically linked, to a carrier for delivery of said fucosylated MMO.
66. A fucosylated MMO for use according to embodiment 65, wherein said linker is a ceramide-based carrier or a polypeptide-based carrier, preferably a ceramide-based carrier.
67. A fucosylated MMO for use according to embodiment 66, wherein said ceramide-based carrier is selected from a list consisting of d18: 1/16:0, t18: 0-16:0, t18: 0-h16: 0, t18: 0-h22: 0 and t18: 0-h24: 0.
68. A fucosylated MMO for use according to any one of embodiments 1 to 67, wherein said fucosylated MMO is encapsulated.
69. A method for preventing and/or treating pneumonia in a farmed animal or a domesticated animal, preferably a farmed animal, wherein said method comprises administering a fucosylated MMO as defined in any one of embodiments 1 to 68.
70. A method according to embodiment 69, wherein said pneumonia is acute pneumonia.
71. A method according to embodiment 69 or 70, wherein said pneumonia is shipping fever pneumonia.
72. A method according to embodiment 69 or 70, wherein said pneumonia is brooder pneumonia.
73. A method according to any one of embodiments 69 to 72, wherein said animal is as defined in any one of embodiments 5 to 7.
74. A method according to any one of embodiments 69 to 73, wherein said method comprises administering an effective amount of said fucosylated MMO to said animal, preferably wherein said fucosylated MMO is administered as defined in any one of embodiments 20 to 33.
75. Use of a fucosylated MMO as defined in any one of embodiments 1 to 68 for the manufacture of a medicament for preventing and/or treating pneumonia in a farmed animal or a domesticated animal, preferably a farmed animal.
76. Use according to embodiment 75, wherein said pneumonia is acute pneumonia.
77. Use according to embodiment 75 or 76, wherein said pneumonia is shipping fever pneumonia.
78. Use according to embodiment 75 or 76, wherein said pneumonia is brooder pneumonia.
79. Use according to any one of embodiments 75 to 78, wherein said animal is as defined in any one of embodiments 5 to 7.
80. Use according to any one of embodiments 75 to 79, wherein said method comprises administering an effective amount of said fucosylated MMO to said animal, preferably wherein said fucosylated MMO is administered as defined in any one of embodiments 20 to 33.

In addition, the present invention preferably relates to the following specific embodiments:
1. A composition comprising a fucosylated mammalian milk oligosaccharide (MMO), wherein said composition is for use in a method for preventing and/or treating pneumonia in a farmed or domesticated animal, preferably a farmed animal.
2. A composition for use according to embodiment 1, wherein said pneumonia is acute pneumonia.
3. A composition for use according to embodiment 1 or 2, wherein said pneumonia is shipping fever pneumonia.
4. A composition for use according to embodiment 1 or 2, wherein said pneumonia is brooder pneumonia.
5. A composition for use according to any one of embodiments 1 to 4, wherein said farmed animal or domesticated animal is selected from the list consisting of bovine, pig, sheep, goat, horse, camelid (preferably llama or alpaca), dog, cat, rabbit and poultry; more preferably selected from the list consisting of bovine, pig, sheep, goat, horse, camelid (preferably llama or alpaca) and poultry; even more preferably selected from the list consisting of bovine, sheep, goat, horse, camelid (preferably llama or alpaca) and poultry; even more preferably selected from the list consisting of bovine, horse and poultry; most preferably said animal is bovine.
6. A composition for use according to any one of embodiments 1 to 5, wherein said animal is a non-adult animal.
7. A composition for use according to embodiment 6, wherein said farmed animal or domesticated animal is selected from the list consisting of a calf, piglet, lamb, kid, foal, cria, puppy, kitten, kit and a chick; more preferably selected from the list consisting of calf, piglet, lamb, kid, foal, cria and chick; even more preferably selected from the list consisting of a calf, lamb, kid, foal, cria and a chick; even more preferably selected from the list consisting of a calf, foal and chick; most preferably said animal is a calf.
8. A composition for use according to any one of embodiments 1 to 7, wherein said fucosylated MMO consists of 3-8, preferably 3-7, more preferably 3-6, even more preferably 3-5, monosaccharides.
9. A composition for use according to any one of embodiments 1 to 8, wherein said fucosylated MMO is a neutral oligosaccharide.
10. A composition for use according to any one of embodiments 1 to 9, wherein said fucosylated MMO comprises a fucose that is linked to a monosaccharide in an alpha-1,2-, alpha-1,3- or alpha-1,4-linkage, preferably an alpha-1,2- or an alpha-1,3-linkage, more preferably an alpha-1,2-linkage, and wherein said monosaccharide is preferably selected from glucose, N-acetylglucosamine and galactose, more preferably said monosaccharide is glucose or galactose, even more preferably said monosaccharide is galactose.
11. A composition for use according to any one of embodiments 1 to 10, wherein said fucosylated MMO comprises a lactose, a lacto-N-biose (LNB) or N-acetyllactosamine (LacNAc) at its reducing end, preferably said fucosylated oligosaccharide comprises lactose or LacNAc at is reducing end, more preferably said fucosylated oligosaccharide comprises lactose at its reducing end.
12. A composition for use according to any one of embodiments 1 to 11, wherein said fucosylated MMO is selected from the list consisting of 2′-fucosyllactose (2′FL), 3-fucosyllactose (3-FL), difucosyllactose (diFL), 2′-fucosyl-N-acetyllactosamine (2′FlacNAc), difucosyl-N-acetyllactosamine (diFLacNAc), 3-fucosyl-N-acetyllactosamine (3FlacNAc), 2′-fucosyllacto-N-biose (2′FLNB), 4-fucosyllacto-N-biose (4FLNB), difucosyllacto-N-biose (diFLNB), lacto-N-fucopentaose I (LNFP I), blood group A antigen hexaose type 1 (GalNAc-LNFP I), blood group B antigen hexaose type 1 (Gal-LNFP I), lacto-N-fucopentaose II (LNFP II), lacto-N-fucopentaose III (LNFP III), lacto-N-fucopentaose V (LNFP V), lacto-N-difucohexaose I (LNDFH I), lacto-N-difucohexaose II (LNDFH II), lewis b-lewis x, monofucosyllacto-N-hexaose III (MFLNH III), difucosyllacto-N-hexaose (a) (DFLNH (a)), difucosyllacto-N-hexaose (DFLNH), trifucosyllacto-N-hexaose (TFLNH), lacto-N-neofucopentaose I (LNnFP I), lacto-N-neofucopentaose V (LNnFP V, LNFP VI), and lacto-N-neodifucohexaose (LNnDFH); more preferably selected from the list consisting of 2′FL, diFL, LNFP I, GalNAc-LNFP I, Gal-LNFP I, LNDFH I, lewis b-lewis x, DFLNH (a), TFLNH, LNnFP I, 2′FLNB, diFLNB, 2′FlacNAc and diFLacNAc; even more preferably selected from the list consisting of 2′FL, diFL, LNFP I, LNnFP I, 2′FLNB, diFLNB, 2′FlacNAc and diFLacNAc; even more preferably selected from the list consisting of 2′FL, diFL, 2′FLNB, diFLNB, 2′FlacNAc and diFLacNAc; even more preferably selected from the list consisting of 2′FL, 2′FLNB and 2′FlacNAc; even more preferably wherein said fucosylated MMO is 2′FL or diFL, most preferably wherein said fucosylated MMO is 2′FL.
13. A composition for use according to any one of embodiments 1 to 12, wherein said fucosylated MMO is selected from the list consisting of 2′FL, diFL, LNFP I, LNnFP I, 2′FLNB, diFLNB, 2′FlacNAc and diFLacNAc.
14. A composition for use according to any one of embodiments 1 to 13, wherein said fucosylated MMO is 2′-fucosyllactose (2′FL).
15. A composition for use according to any one of embodiments 1 to 14, wherein said fucosylated MMO is present at 0.001-1.000 wt. %, preferably 0.001-0.750 wt. %, more preferably 0.001-0.500 wt. %, even more preferably 0.001-0.400 wt. %, even more preferably 0.005-0.400 wt. %, even more preferably 0.005-0.300 wt. %, even more preferably 0.005-0.250 wt. %, most preferably 0.005-0.100 wt. %, of the total weight of the composition.
16. A composition for use according to any one of embodiments 1 to 15, wherein said fucosylated MMO is present at 0.001-0.250 wt. %, preferably 0.001-0.200 wt. %, more preferably 0.001-0.100 wt. %, even more preferably 0.001-0.020 wt. %, even more preferably 0.001-0.010 wt. %, even more preferably 0.002-0.010 wt. %, even more preferably 0.003-0.010 wt. %, even more preferably 0.003-0.0075 wt. %, most preferably 0.003-0.006 wt. %, of the total weight of the composition; preferably wherein said animal is a bovine or a horse, more preferably a calf or a foal, even more preferably a calf.
17. A composition for use according to any one of embodiments 1 to 15, wherein said fucosylated MMO is present at 0.010-1.000 wt. %, preferably 0.010-0.750 wt. %, more preferably 0.010-0.500 wt. %, even more preferably 0.010-0.400 wt. %, even more preferably 0.050-0.400 wt. %, even more preferably 0.100-0.400 wt. %, most preferably 0.100-0.300 wt. %, of the total weight of the composition; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
18. A composition for use according to any one of embodiments 1 to 17, wherein said method comprises administering an effective amount of said fucosylated MMO.
19. A composition for use according to any one of embodiments 1 to 18, wherein said fucosylated MMO is administered to said animal at least once, preferably at least twice, between birth (0 weeks) and 5 weeks of age, preferably between birth and 4 weeks of age, more preferably between birth and 3 weeks of age.
20. A composition for use according to any one of embodiments 1 to 19, wherein said fucosylated MMO is administered to said animal at least once, preferably at least twice, more preferably daily:
in the three weeks, preferably two weeks, more preferably one week, preceding the day of transporting said animal; and/or
on the day of transporting said animal; and/or
in the three weeks, preferably two weeks, more preferably one week, after the day of transporting said animal.
21. A composition for use according to any one of embodiments 1 to 20, wherein said fucosylated MMO is administered to said animal for at least 1 week, preferably at least 2 weeks, more preferably at least 3 weeks, even more preferably at least 4 weeks, most preferably at least 6 weeks.
22. A composition for use according to any one of embodiments 1 to 21, wherein said fucosylated MMO is administered to said animal for ≤32 weeks, preferably ≤28 weeks, more preferably ≤24 weeks, even more preferably ≤20 weeks, even more preferably ≤16 weeks, even more preferably ≤12 weeks, most preferably ≤6 weeks.
23. A composition for use according to any one of embodiments 1 to 22, wherein said fucosylated MMO is administered to said animal for 1-36, preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks.
24. A composition for use according to any one of embodiments 1 to 23, wherein said fucosylated MMO is administered to said animal for:
   if subject is bovine: 1-36, preferably 1-32, more preferably 1-30, even more preferably 1-28, even more preferably 1-24, even more preferably 1-18, even more preferably 1-12, even more preferably 2-12, most preferably 3-12, consecutive weeks;
   if subject is a pig: 1-18, preferably 1-12, more preferably 1-8, even more preferably 1-6, even more preferably 1-4, most preferably 2-4, consecutive weeks;
   if subject is a sheep: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a goat: 1-36, preferably 1-32, more preferably 1-30, even more preferably 1-28, even more preferably 1-24, even more preferably 1-18, even more preferably 1-12, even more preferably 1-8, even more preferably 1-6, even more preferably 1-4, most preferably 2-4, consecutive weeks;
   if subject is a horse: 1-18, preferably 1-12, more preferably 1-8, even more preferably 1-6, even more preferably 1-4, most preferably 2-4, consecutive weeks;
   if subject is a camelid: 1-18, preferably 1-12, more preferably 1-8, even more preferably 1-6, even more preferably 1-4, most preferably 2-4, consecutive weeks;
   if subject is a rabbit: 1-12, preferably 1-8, more preferably 1-6, even more preferably 1-4, most preferably 2-4, consecutive weeks;
   if subject is a dog: 1-12, preferably 1-8, more preferably 1-6, even more preferably 1-4, most preferably 2-4, consecutive weeks;
   if subject is a cat: 1-12, preferably 1-8, more preferably 1-6, even more preferably 1-4, most preferably 2-4, consecutive weeks;
   if subject is poultry: 1-8, preferably 1-6, more preferably 1-4, most preferably 2-4, consecutive weeks.
25. A composition for use according to any one of embodiments 1 to 24, wherein said fucosylated MMO is administered at least once a week, preferably at least once every 3 days, more preferably at least once every 2 days, most preferably at least once daily.
26. A composition for use according to any one of embodiments 1 to 25, wherein said fucosylated MMO is administered at a daily dose of 0.0001-15.0 g, preferably 0.0001-10.0 g, more preferably 0.0001-5.0 g, even more preferably 0.0001-2.0 g, even more preferably 0.0001-1.0 g, even more preferably 0.0005-1.0 g, most preferably 0.0010-1.0 g.
27. A composition for use according to any one of embodiments 1 to 25, wherein said fucosylated MMO is administered at a daily dose of 0.01-15.0 g, preferably 0.01-10.0 g, more preferably 0.01-5.0 g, even more preferably 0.01-2.0 g, even more preferably 0.01-1.0 g, even more preferably 0.015-1.0 g, most preferably 0.02-1.0 g.
28. A composition for use according to any one of embodiments 1 to 25, wherein said fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein said animal is a bovine or a horse, more preferably a calf or a foal, even more preferably a calf.
29. A composition for use according to any one of embodiments 1 to 25, wherein said fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
30. A composition for use according to any one of embodiments 1 to 29, wherein said fucosylated MMO is administered at a daily dose of 0.01-150.0 mg, preferably 0.01-125.0 mg, more preferably 0.01-100.0 mg, even more preferably 0.05-100.0 mg, even more preferably 0.1-100.0 mg, most preferably 0.1-80.0 mg, per kg bodyweight of said animal.
31. A composition for use according to any one of embodiments 1 to 29, wherein said fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of said animal; preferably wherein said animal is a bovine or a horse, more preferably a calf or a foal, even more preferably a calf.
32. A composition for use according to any one of embodiments 1 to 29, wherein said fucosylated MMO is administered at a daily dose of 0.1-150.0 mg, preferably 0.1-125.0 mg, more preferably 1.0-125.0 mg, even more preferably 5.0-125.0 mg, even more preferably 5.0-100.0 mg, even more preferably 5.0-80.0 mg, most preferably 10.0-80.0 mg, per kg bodyweight of said animal; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
33. A composition for use according to any one of embodiments 1 to 32, wherein said composition further comprises a second mammalian milk oligosaccharide (MMO), preferably wherein said second mammalian milk oligosaccharide is a sialylated MMO, more preferably wherein said second mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.
34. A composition for use according to embodiment 33, wherein said second MMO is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably said second MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably said second MMO is selected from the list consisting of 3′SL, 6′SL, 3′SLNB, 6′SLNB, 3′SLacNAc and 6′SLacNAc; most preferably said second MMO is 3′-sialyllactose (3′SL) or 6′-sialyllactose (6′SL).
35. A composition for use according to embodiment 33 or 34, wherein said second MMO is 3′-sialyllactose (3′SL) or 6′-sialyllactose (6′SL).
36. A composition for use according to any one of embodiments 33 to 35, wherein the amount of said fucosylated MMO in said composition is lower than the amount of said second MMO, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said second MMO in said composition.
37. A composition for use according to any one of embodiments 33 to 35, wherein the amount of said fucosylated MMO in said composition is lower than the amount of said second MMO, preferably said amount of said fucosylated MMO is 1.1 to 10.0, preferably 1.5-7.5, more preferably 2.5-7.5, most preferably 4.0-7.0, times lower than the amount of said second MMO in said composition.
38. A composition for use according to any one of embodiments 33 to 35, wherein the amount of said second MMO in said composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO in said composition.
39. A composition for use according to any one of embodiments 1 to 38, wherein said composition further comprises a third mammalian milk oligosaccharide (MMO), preferably wherein said third mammalian milk oligosaccharide is a sialylated MMO, more preferably wherein said third mammalian milk oligosaccharide is a sialylated MMO comprising a lactose, a lacto-N-biose (LNB) or N-acetyllactosamine (LacNAc) at its reducing end, even more preferably said sialylated MMO comprises lactose or LacNAc at is reducing end, most preferably said sialylated MMO comprises lactose at its reducing end.
40. A composition for use according to embodiment 39, wherein said thrid MMO is selected from the list consisting of 3′-sialyllactose (3′SL), 6′-sialyllactose (6′SL), 3,6-disialyllactose, 6,6′-disialyllactose, 8,3-disialyllactose, 3'S-2′FL, 6'S-2′FL, 3'S-3-FL, 6'S-3-FL, LST a, LST b, LST c, LST d, 3′-sialyllacto-N-biose (3′SLNB), 6′-sialyllacto-N-biose (6′SLNB), sialyl Lewis a, 3′-sialyllactosamine (3′SLacNAc), 6′-sialyllactosamine (6′SLacNAc) and sialyl Lewis x; preferably said third MMO is selected from the list consisting of 3′SL, 6′SL, LST a, LST b, LST c, LST d, 3′SLNB, 6′SLNB, sialyl Lewis a, 3′SLacNAc, 6′SLacNAc and sialyl Lewis x; more preferably said third MMO is selected from the list consisting of 3′SL, 6′SL, 3′SLNB, 6′SLNB, 3′SLacNAc and 6′SLacNAc; most preferably said third MMO is 6′-sialyllactose (6′SL) or 3′-sialyllactose (3′SL).
41. A composition for use according to embodiment 39 or 40, wherein said third MMO is 6′-sialyllactose (6′SL) or 3′-sialyllactose (3′SL).
42. A composition for use according to any one of embodiments 39 to 41, wherein the amount of said fucosylated MMO in said composition is lower than the amount of said third MMO, preferably said amount of said fucosylated MMO is 1.1 to 15.0, more preferably 1.5 to 15.0, even more preferably 2.5 to 15.0, even more preferably 5.0 to 15.0, even more preferably 5.0 to 12.5, most preferably 5.0 to 10.0, times lower than the amount of said third MMO in said composition.
43. A composition for use according to any one of embodiments 39 to 41, wherein the amount of said fucosylated MMO in said composition is lower than the amount of said third MMO, preferably said amount of said fucosylated MMO is 1.1 to 10.0, preferably 1.5-7.5, more preferably 1.5-5.0, most preferably 2.5-5.0, times lower than the amount of said third MMO in said composition.
44. A composition for use according to any one of embodiments 39 to 41, wherein the amount of said third MMO in said composition is 0.1-30.0, preferably 0.1-25.0, more preferably 0.1-20.0, even more preferably 0.1-15.0, even more preferably 0.2-15.0, even more preferably 0.3-15.0, even more preferably 0.4-15.0, most preferably 0.5-15.0, times the amount of said fucosylated MMO in said composition.
45. A composition for use according to any one of embodiments 39 to 44, wherein the ratio between the amount of said second MMO and the amount of said third MMO in said composition is similar to this ratio as found in the mother's milk of the same species.
46. A composition for use according to any one of embodiments 39 to 45, wherein the amount of said second MMO in said composition is higher than the amount of said third MMO, preferably said amount of said second MMO is 1.1 to 40.0, more preferably 1.1 to 35.0, most preferably 1.5 to 35.0, times the amount of said third MMO in said composition.
47. A composition for use according to any one of embodiments 39 to 45, wherein the amount of said second MMO in said composition is higher than the amount of said third MMO, preferably said amount of said second MMO is 1.1 to 6.0, more preferably 1.1 to 5.0, even more preferably 1.25 to 4.0, even more preferably 1.25-3.0, even more preferably 1.5 to 3.0, most preferably 1.5-2.5, times higher than the amount of said third MMO in said composition.
48. A composition for use according to any one of embodiments 1 to 47, wherein said composition does not comprise lacto-N-tetraose (LNT).
49. A composition for use according to any one of embodiments 1 to 48, wherein said composition does not comprise lacto-N-neotetraose (LNnT).
50. A composition for use according to any one of embodiments 1 to 49, wherein said composition does not comprise N-acetyllactosamine.
51. A composition for use according to any one of embodiments 1 to 50, wherein said composition does not comprise an oligosaccharide containing N-acetyllactosamine, preferably does not comprise a saccharide containing N-acetyllactosamine.
52. A composition for use according to any one of embodiments 1 to 51, wherein said composition does not comprise a fructo-oligosaccharide (FOS).
53. A composition for use according to any one of embodiments 1 to 52, wherein said composition does not comprise a galacto-oligosaccharide (GOS).
54. A composition for use according to any one of embodiments 1 to 53, wherein said composition does not comprise an isomaltooligosaccharide.
55. A composition for use according to any one of embodiments 1 to 54, wherein said composition does not comprise a neutral non-fucosylated MMO.
56. A composition for use according to any one of embodiments 1 to 32 and 48 to 55, wherein said composition does not comprise a further mammalian milk oligosaccharide.
57. A composition for use according to any one of embodiments 1 to 56, wherein said composition further comprises a microbial strain which does not use, preferably is not capable to use, said fucosylated MMO as carbon source.
58. A composition for use according to any one of embodiments 1 to 56, wherein said composition does not comprise a microbial strain.
59. A composition for use according to any one of embodiments 1 to 58, wherein said composition is not mother's milk.
60. A composition for use according to any one of embodiments 1 to 59, wherein said composition is a synthetic composition.
61. A composition for use according to any one of embodiments 1 to 60, wherein said fucosylated MMO has been produced in vitro and/or ex vivo by a cell, preferably a single cell, wherein said cell is preferably chosen from the list consisting of a microorganism, a plant cell, an animal cell and a protozoan cell.
62. A composition for use according to any one of embodiments 1 to 61, wherein said fucosylated MMO is linked, preferably chemically linked, to a carrier for delivery of said fucosylated MMO.
63. A composition for use according to embodiment 62, wherein said linker is a ceramide-based carrier or a polypeptide-based carrier, preferably a ceramide-based carrier.
64. A composition for use according to embodiment 63, wherein said ceramide-based carrier is selected from a list consisting of d18: 1/16:0, t18: 0-16:0, t18: 0-h16: 0, t18: 0-h22: 0 and t18: 0-h24: 0.
65. A composition for use according to any one of embodiments 1 to 64, wherein said fucosylated MMO is encapsulated.
66. A composition for use according to any one of embodiments 1 to 65, wherein said composition is a pharmaceutical composition, optionally further comprising a pharmaceutically acceptable carrier, filler, preservative, solubilizer, diluent, excipient, salt, adjuvant and/or solvent.
67. A composition for use according to any one of embodiments 1 to 66, wherein said composition is a nutritional composition, optionally further comprising a feed ingredient and/or a food ingredient, wherein said feed/food ingredient is preferably chosen from the list consisting of:
   a lipid, preferably one or more selected from the list consisting of an oil, fat, ester, monoglyceride, diglyceride, triglyceride and free fatty acid;
   a vitamin, preferably one or more selected from the list consisting of vitamin A, vitamin B, vitamin C, vitamin D, vitamin E and vitamin H, or a derivate thereof;
   an amino acid compound;
   a trace element;
   a mineral, preferably one or more selected from the list consisting of calcium, phosphorus, magnesium, iron, zinc, manganese, copper, sodium, potassium, molybdenum, chromium, selenium and chloride;
   an antioxidant;
   a prebiotic agent, preferably one or more selected from the list consisting of GOS (galacto-oligosaccharide), FOS (fructo-oligosaccharide), inulin and resistant starch;
   a carbohydrate;
   an antimicrobial agent; and/or
   a protein.
68. A composition for use according to embodiment 67, wherein said nutritional composition is selected from a list consisting of a milk replacer, creep feed, prestarter diet, starter diet, pre-weaning feed, weaning feed and post-weaning feed, preferably selected form the list consisting of a milk replacer, starter diet, creep feed and post-weaning feed, more preferably said nutritional composition is a milk replacer or starter diet, most preferably a milk replacer.
69. A method for preventing and/or treating pneumonia in a farmed animal or a domesticated animal, preferably a farmed animal, wherein said method comprises administering a composition as defined in any one of embodiments 1 to 68.
70. A method according to embodiment 69, wherein said pneumonia is acute pneumonia.
71. A method according to embodiment 69 or 70, wherein said pneumonia is shipping fever pneumonia.
72. A method according to embodiment 69 or 70, wherein said pneumonia is brooder pneumonia.
73. A method according to any one of embodiments 69 to 72, wherein said animal is as defined in any one of embodiments 5 to 7.
74. A method according to any one of embodiments 69 to 73, wherein said method comprises administering an effective amount of said fucosylated MMO to said animal, preferably wherein said fucosylated MMO is administered as defined in any one of embodiments 19 to 32.
75. Use of a composition as defined in any one of embodiments 1 to 68 for the manufacture of a medicament for preventing and/or treating pneumonia in an animal, wherein said animal is a farmed animal or a domesticated animal, preferably a farmed animal.
76. Use according to embodiment 75, wherein said pneumonia is acute pneumonia.
77. Use according to embodiment 75 or 76, wherein said pneumonia is shipping fever pneumonia.
78. Use according to embodiment 75 or 76, wherein said pneumonia is brooder pneumonia.
79. Use according to any one of embodiments 75 to 78, wherein said animal is as defined in any one of embodiments 5 to 7.
80. Use according to any one of embodiments 75 to 79, wherein said method comprises administering an effective amount of said fucosylated MMO to said animal, preferably wherein said fucosylated MMO is administered as defined in any one of embodiments 19 to 32.

The present invention more preferably relates to the following specific embodiments:
1. A fucosylated mammalian milk oligosaccharide (MMO) for use in a method for preventing and/or treating pneumonia in a farmed or domesticated animal, preferably a farmed animal.
2. A fucosylated MMO for use according to embodiment 1, wherein said method is a method for preventing and/or treating pneumonia by enhancing the T lymphocyte immunity in said animal.
3. A fucosylated MMO for use according to embodiment 1 or 2, wherein said pneumonia is acute pneumonia.
4. A fucosylated MMO for use according to any one of embodiments 1 to 3, wherein said pneumonia is shipping fever pneumonia or brooder pneumonia, preferably shipping fever pneumonia.
5. A fucosylated MMO for use according to any one of embodiments 1 to 4, wherein said farmed animal or domesticated animal is selected from the list consisting of bovine, pig, sheep, goat, horse, donkey, mule, hinny, camelid, dog, cat, rabbit and poultry.
6. A fucosylated MMO for use according to any one of embodiments 1 to 5, wherein said animal is a non-adult animal.
7. A fucosylated MMO for use according to any one of embodiments 1 to 6, wherein said fucosylated MMO comprises a fucose that is linked to a monosaccharide in an alpha-1,2-linkage, and wherein said monosaccharide is galactose.
8. A fucosylated MMO for use according to any one of embodiments 1 to 7, wherein said fucosylated MMO is 2′-fucosyllactose (2′FL).
9. A fucosylated MMO for use according to any one of embodiments 1 to 8, wherein said method does not comprise administering lacto-N-tetraose (LNT) or lacto-N-neotetraose (LNnT).
10. A fucosylated MMO for use according to any one of embodiments 1 to 9, wherein said method does not comprise administering an additional MMO.
11. A fucosylated MMO for use according to any one of embodiments 1 to 10, wherein said fucosylated MMO is administered to said animal at least once, preferably at least twice, between birth (0 weeks) and 5 weeks of age, preferably between birth and 4 weeks of age, more preferably between birth and 3 weeks of age.
12. A fucosylated MMO for use according to any one of embodiments 1 to 11, wherein said fucosylated MMO is administered to said animal at least once, preferably at least twice, more preferably daily:
   in the three weeks, preferably two weeks, more preferably one week, preceding the day of transporting said animal; and/or
   on the day of transporting said animal; and/or
   in the three weeks, preferably two weeks, more preferably one week, after the day of transporting said animal.
13. A fucosylated MMO for use according to any one of embodiments 1 to 12, wherein said fucosylated MMO is administered at a daily dose of 0.01-1.0 g, preferably 0.01-0.75 g, more preferably 0.01-0.65 g, even more preferably 0.01-0.60 g, even more preferably 0.01-0.50 g, even more preferably 0.015-0.50 g, even more preferably 0.015-0.40 g, even more preferably 0.015-0.30 g, even more preferably 0.015-0.20 g, most preferably 0.015-0.10 g; preferably wherein said animal is a bovine or a horse, more preferably a calf or a foal, even more preferably a calf.
14. A fucosylated MMO for use according to any one of embodiments 1 to 12, wherein said fucosylated MMO is administered at a daily dose of 0.01-5.0 g, preferably 0.1-2.5 g, more preferably 0.1-1.5 g, even more preferably 0.1-1.2 g, even more preferably 0.2-1.2 g, even more preferably 0.3-1.2 g, even more preferably 0.4-1.2 g, most preferably 0.5-1.2 g; preferably wherein said animal is a pig, a sheep, a goat or a camelid (preferably llama or alpaca), more preferably wherein said animal is a piglet, a lamb, a kid or a cria, even more preferably wherein said animal is a piglet, a lamb or a kid, most preferably wherein said animal is a piglet.
15. A fucosylated MMO for use according to any one of embodiments 1 to 14, wherein said fucosylated MMO is administered at a daily dose of 0.1-50.0 mg, preferably 0.1-30.0 mg, more preferably 0.1-25.0 mg, even more preferably 0.1-20.0 mg, even more preferably 0.1-15.0 mg, even more preferably 0.1-10.0 mg, even more preferably 0.25-10.0 mg, most preferably 0.5-10.0 mg, per kg bodyweight of said animal; preferably wherein said animal is a bovine or a horse, more preferably a calf or a foal, even more preferably a calf.
16. A fucosylated MMO for use according to any one of embodiments 1 to 15, wherein said fucosylated MMO is part of a composition.
17. A fucosylated MMO for use according to embodiment 16, wherein said composition is not mother's milk.
18. A fucosylated MMO for use according to embodiment 16 or 17, wherein said composition is a pharmaceutical composition, optionally further comprising a pharmaceutically acceptable carrier, filler, preservative, solubilizer, diluent, excipient, salt, adjuvant and/or solvent.
19. A fucosylated MMO for use according to any one of embodiments 16 to 18, wherein said composition is a nutritional composition and selected from a list consisting of a milk replacer, creep feed, prestarter diet, starter diet, pre-weaning feed, weaning feed and post-weaning feed, preferably selected form the list consisting of a milk replacer, starter diet, creep feed and post-weaning feed, more preferably said nutritional composition is a milk replacer or starter diet, most preferably a milk replacer.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various aspects and embodiments of the invention disclosed herein are to be understood not only in the order and context specifically described in this specification, but to include any order and any combination thereof. Each embodiment as identified herein may be combined together unless otherwise indicated. All publications, patents and patent applications cited in this specification are herein incorporated by reference in their entirety to the same extent as if each individual publication, patent or patent application was specifically and individually indicated to be incorporated by reference in its entirety. Unless specifically stated otherwise, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the invention herein and within the scope of this invention, which is limited only by the claims, construed in accordance with the patent law, including the doctrine of equivalents. In the claims which follow, reference characters used to designate claim steps are provided for convenience of description only, and are not intended to imply any particular order for performing the steps (unless specifically stated otherwise).

In this document and in its claims, the verbs “to comprise”, “to have” and “to contain”, and their conjugations are used in their non-limiting sense to mean that items following the word are included, but items not specifically mentioned are not excluded. The verb “to consist essentially of” means that e.g. a composition as defined herein may comprise additional component(s) than the ones specifically identified, said additional component(s) not altering the unique characteristic of the invention.

Throughout the document and claims, unless specifically stated otherwise, the verbs “to comprise”, “to have” and “to contain”, and their conjugations, may be preferably replaced by “to consist” (and its conjugations) or “to consist essentially of” (and its conjugations) and vice versa. In addition, reference to an element by the indefinite article “a” or “an” does not exclude the possibility that more than one of the element is present, unless the context clearly requires that there be one and only one of the elements.

The indefinite article “a” or “an” thus usually means “at least one”. The word “about” or “approximately” or “around” when used in association with a numerical value (e.g. about 10) preferably means that the value may be the given value (of 10) more or less 10%, preferably 5%, more preferably 1% of the value. Throughout the description and claims, unless specifically stated otherwise, the expression “from x to y”, wherein x and y represent numerical values, refers to a range of numerical values wherein both x and y are included and wherein x represents the lowest value and y represents the highest value. Hence, both x and y are included in the range.

The terms “LNT II”, “LNT-II”, “LN3”, “lacto-N-triose II”, “lacto-N-triose II”, “lacto-N-triose”, “lacto-N-triose” and “GlcNAc-β1,3-Gal-β1,4-Glc” are used interchangeably.

The terms “LNT”, “lacto-N-tetraose”, “lacto-N-tetraose” and “Gal-β1,3-GlcNAc-β1,3-Gal-β1,4Glc” are used interchangeably.

The terms “LNnT”, “lacto-N-neotetraose”, “lacto-N-neotetraose”, “neo-LNT” and “Galβ1-4GlcNAcβ1-3Galβ1-4Glc” are used interchangeably.

The terms “2′ fucosyllactose”, “2′-fucosyllactose”, “alpha-1,2-fucosyllactose”, “alpha 1,2 fucosyllactose”, “α-1,2-fucosyllactose”, “a 1,2 fucosyllactose”, “Fuc-α1,2-Gal-β1,4-Glc”, 2FL″ and “2′FL” are used interchangeably.

The terms “3-fucosyllactose”, “alpha-1,3-fucosyllactose”, “alpha 1,3 fucosyllactose”, “α-1,3-fucosyllactose”, “α1,3 fucosyllactose”, “Gal-β1,4-(Fuc-α1,3-) Glc”, 3FL″ and “3-FL” are used interchangeably.

The terms “difucosyllactose”, “di-fucosyllactose”, “lactodifucotetraose”, “2′,3-difucosyllactose”, “2′,3 difucosyllactose”, “α-2′,3-fucosyllactose”, “α2′,3 fucosyllactose, “Fuc-α1,2-Gal-β1,4-(Fuc-α1,3-) Glc”, “DFLac”, 2′,3 diFL”, “DFL”, “DiFL” and “diFL” are used interchangeably.

The terms “LNFP-I”, “lacto-N-fucopentaose I”, “LNFP I”, “LNF I OH type I determinant”, “LNF I”, “LNF1”, “LNF 1”, “Blood group H antigen pentaose type 1” and “Fuc-α1,2-Gal-β1,3-GlcNAc-β1,3-Gal-β1,4-Glc” are used interchangeably.

The terms “GalNAc-LNFP-I”, “blood group A antigen hexaose type I”, and “GalNAc-α1,3-(Fuc-α1,2)-Gal-β1,3-GlcNAc-β1,3-Gal-β1,4-Glc” are used interchangeably.

The terms “Gal-LNFP-I”, “blood group B antigen hexaose type I” and “Gal-x1,3-(Fuc-α1,2)-Gal-β1,3-GlcNAc-β1,3-Gal-β1,4-Glc” are used interchangeably.

The terms “LNFP-II”, “lacto-N-fucopentaose II” and “Gal-β1,3-(Fuc-α1,4)-GlcNAc-β1,3-Gal-β1,4-Glc” are used interchangeably.

The terms “LNFP-III”, “lacto-N-fucopentaose III” and “Gal-β1,4-(Fuc-α1,3)-GlcNAc-β1,3-Gal-β1,4-Glc” are used interchangeably.

The terms “LNFP-V”, “lacto-N-fucopentaose V” and “Gal-β1,3-GlcNAc-β1,3-Gal-β1,4-(Fuc-α1,3)-Glc” are used interchangeably.

The terms “LNDFH I”, “Lacto-N-difucohexaose I”, “LNDFH-I”, “LDFH I”, “Leb-lactose”, “Lewis-b hexasaccharide” and “Fuc-α1,2-Gal-β1,3-[Fuc-α1,4]-GlcNAc-β1,3-Gal-β1,4-Glc” are used interchangeably. The terms “LNDFH II”, “Lacto-N-difucohexaose II”, “Lewis α-Lewis x”, “LDFH II” and “Fuc-α1,4-(Gal-β1,3)-GlcNAc-β1,3-Gal-β1,4-(Fuc-α1,3)-Glc” are used interchangeably.

The terms “lewis b-lewis x” and “Fucα1,4-[Fuc-α1,2-Galβ1,3]-GlcNAc-β1,3-Gal-β1,4-[Fuc-α1,3]-Glc are used interchangeably.

The terms “MFLNH III”, “monofucosyllacto-N-hexaose-III” and “Gal-β1,4-[Fuc-α1,3]-GlcNAc-β1,6-[Gal-β1,3-GlcNAc-β1,3]-Gal-β1,4-Glc” are used interchangeably.

The terms “DFLNH (a)”, “difucosyllacto-N-hexaose (a)” and “Gal-β1,4-[Fuc-α1,3]-GlcNAc-β1,6-[Fuc-α1,2-Gal-β1,3-GlcNAc-β1,3]-Gal-β1,4-Glc” are used interchangeably.

The terms “DFLNH”, “difucosyllacto-N-hexaose” and “Gal-β1,4-[Fuc-α1,3]-GlcNAc-β1,6-[Fuc-α1,4-[Gal-β1,3]-GlcNAc-β1,3]-Gal-β1,4-Glc” are used interchangeably.

The terms “TFLNH”, “trifucosyllacto-N-hexaose” and “Gal-β1,4-[Fuc-α1,3]-GlcNAc-β1,6-[Fuc-α1,4-[Fuc-α1,2-Gal-β1,3]-GlcNAc-β1,3]-Gal-β1,4-Glc” are used interchangeably.

The terms “LNnFP I”, “Lacto-N-neofucopentaose I” and “Fuc-α1,2-Gal-β1,4-GlcNAc-β1,3-Gal-β1,4-Glc” are used interchangeably.

The terms “LNFP-VI”, “LNnFP V”, “lacto-N-neofucopentaose V” and “Gal-β1,4-GlcNAc-β1,3-Gal-β1,4-(Fuc-α1,3)-Glc” are used interchangeably.

The terms “LNnDFH”, “Lacto-N-neoDiFucohexaose”, “Lewis x hexaose” “Gal-β1,4-(Fuc-α1,3)-GlcNAc-β1,3-Gal-β1,4-(Fuc-α1,3)-Glc” are used interchangeably.

The terms “2′-fucosyllacto-N-biose”, “2′FLNB” and “Fuc-α1,2-Gal-β1,3-GlcNAc” are used interchangeably.

The terms “4-fucosyllacto-N-biose”, “4FLNB” and “Fuc-α1,4-[Gal-β1,3-]GlcNAc” are used interchangeably.

The terms “difucosyllacto-N-biose”, “diFLNB” and “Fuc-α1,4-[Fuc-α1,2-Gal-β1,3-]GlcNAc” are used interchangeably.

The terms “2′-fucosyl-N-acetyllactosamine”, “2′FlacNAc” and “Fuc-α1,2-Gal-β1,4-GlcNAc” are used interchangeably.

The terms “3-fucosyl-N-acetyllactosamine”, “3FlacNAc” and “Gal-β1,4-(Fuc-α1,3-) GlcNAc” are used interchangeably.

The terms “difucosyl-N-acetyllactosamine”, “diFlacNAc” and “Fuc-α1,2-Gal-β1,4-[Fuc-α1,3-]GlcNAc” are used interchangeably.

The terms “3′ sialyllactose”, “3′-sialyllactose”, “alpha-2,3-sialyllactose”, “alpha 2,3 sialyllactose”, “α-2,3-sialyllactose”, “a 2,3 sialyllactose”, “3SL”, “Sia-α2,3-Gal-β1,4-Glc” and “3′SL” are used interchangeably. The terms “6′ sialyllactose”, “6′-sialyllactose”, “alpha-2,6-sialyllactose”, “alpha 2,6 sialyllactose”, “α-2,6-sialyllactose”, “a 2,6 sialyllactose”, “6SL”, “Sia-x2,6-Gal-β1,4-Glc” and “6′SL” are used interchangeably.

The terms “3,6-disialyllactose” and “Neu5Ac-α2,3-Neu5Ac-α2,6-Gal-β1,4-Glc” are used interchangeably.

The terms “6,6′-disialyllactose” and “Neu5Ac-α2,6-Neu5Ac-α2,6-Gal-β1,4-Glc” are used interchangeably.

The terms “8,3-disialyllactose” and “Neu5Ac-α2,8-Neu5Ac-α2,3-Gal-β1,4-Glc” are used interchangeably.

The terms “3'S-2′FL” and “Neu5Ac-α2,3-[Fuc-α1,2-]Gal-β1,4-Glc” are used interchangeably.

The terms “6'S-2′FL” and “Neu5Ac-α2,6-[Fuc-α1,2-]Gal-β1,4-Glc” are used interchangeably.

The terms “3'S-3-FL” and “Neu5Ac-α2,3-Gal-β1,4-[Fuc-α1,3]Glc” are used interchangeably.

The terms “6'S-3-FL” and “Neu5Ac-α2,6-Gal-β1,4-[Fuc-α1,3]Glc” are used interchangeably.

The terms “LSTa”, “LS-Tetrasaccharide a”, “Sialyl-lacto-N-tetraose a”, “sialyllacto-N-tetraose a” and “Neu5Ac-α2,3-Gal-b1,3-GlcNAc-b1,3-Gal-b1,4-Glc” are used interchangeably.

The terms “LSTb”, “LS-Tetrasaccharide b”, “Sialyl-lacto-N-tetraose b”, “sialyllacto-N-tetraose b” and “Gal-b1,3-(Neu5Ac-α2,6)-GlcNAc-b1,3-Gal-b1,4-Glc” are used interchangeably.

The terms “LSTc”, “LS-Tetrasaccharide c”, “Sialyl-lacto-N-tetraose c”, “sialyllacto-N-tetraose c”, “sialyllacto-N-neotetraose c” and “Neu5Ac-α2,6-Gal-b1,4-GlcNAc-b1,3-Gal-b1,4-Glc” are used interchangeably.

The terms “LSTd”, “LS-Tetrasaccharide d”, “Sialyl-lacto-N-tetraose d”, “sialyllacto-N-tetraose d”, “sialyllacto-N-neotetraose d” and “Neu5Ac-α2,3-Gal-b1,4-GlcNAc-b1,3-Gal-b1,4-Glc” are used interchangeably.

The terms “3′-sialyllacto-N-biose”, “3′SLNB” and “Neu5Ac-α2,3-Gal-b1,3-GlcNAc” are used interchangeably.

The terms “6′-sialyllacto-N-biose”, “6′SLNB” and “Neu5Ac-α2,6-Gal-b1,3-GlcNAc” are used interchangeably.

The terms “monofucosylmonosialyllacto-N-octaose”, “sialyl Lewis a”, “sialyl Lea”, “5-acetylneuraminyl-(2-3)-galactosyl-(1-3)-(fucopyranosyl-(1-4))-N-acetylglucosamine” and “Neu5Ac-α2,3-Gal-β1,3-[Fuc-α1,4]-GlcNAc” are used interchangeably.

The terms “3′-sialyllactosamine”, “3′SLacNAc” and “Neu5Ac-α2,3-Gal-b1,4-GlcNAc” are used interchangeably.

The terms “6′-sialyllactosamine”, “6′SLacNAc” and “Neu5Ac-α2,6-Gal-b1,4-GlcNAc” are used interchangeably.

The terms “sialyl Lewis x”, “sialyl Lex”, “5-acetylneuraminyl-(2-3)-galactosyl-(1-4)-(fucopyranosyl-(1-3))-N-acetylglucosamine” and “Neu5Ac-α2,3-Gal-β1,4-[Fuc-α1,3-]GlcNAc” are used interchangeably.

The term “cultivation” refers to the culture medium wherein the cell is cultivated or fermented, the cell itself, and the saccharide(s) that is/are produced by the cell in whole broth, i.e. inside (intracellularly) as well as outside (extracellularly) of the cell.

The term “clarifying” refers to the act of treating an aqueous solution to remove suspended particulates and contaminants from the production process, like e.g. cells, cell components, insoluble metabolites and debris, that could interfere with the eventual purification of the oligosaccharide(s) of interest. Such treatment can be carried out in a conventional manner by centrifugation, flocculation, flocculation with optional ultrasonic treatment, gravity filtration, microfiltration, foam separation or vacuum filtration (e.g., through a ceramic filter which can include a Celite™ filter aid).

### BRIEF DESCRIPTION OF THE DRAWINGS

The following drawings will serve as further illustration and clarification of the present invention and are not intended to be limiting in any way.

FIG. 1. Probability of first pneumonia incidence over time in control (placebo) and 2′-fucosyllactose (2′FL) treated animals of the first trial (veal calves).

FIG. 2. Probability of first pneumonia incidence over time in control (placebo) and 2′-fucosyllactose (2′FL) treated animals of the second trial (rearing calves).

### EXAMPLES

The invention will be described in more detail in the examples. The following examples will serve as further illustration and clarification of the present invention and are not intended to be limiting in any way.

### Example 1: 2′FL

2′-fucosyllactose (2′FL) was recombinantly produced in E. coli and purified as described in examples 10, 13 and 14 of WO 2022/034079, and subsequently spray dried as described in Example 21 of WO 2022/034079 to obtain 2″FL powder (purity 89.6%).

### Example 2: Trials in Calves

### Animals and Diets

Holstein Friesian calves (2-3 week-old, 95% male) were transported from different farms to the experimental facility of Denkavit (Voorthuizen, The Netherlands) where the animals were reared. The experiment was performed in two trial rounds.

### Round 1: Veal Calf

The calves were randomly and gender-equally divided in two groups based on experimental calf milk replacer: group A (placebo-standard calf milk replacer (CMR) powder of the farm) and group B (standard CMR powder supplemented with 0.005% drywt. 2′-fucosyllactose (2′FL, see Example 1). The calf milk replacer was administered as a liquid to the calves (water was added to the CMR powder to obtain 150 gram CMR powder per liter liquid CMR). Each group involved 290 calves, assigned into 12 rows. Each row contained 4 pens, where six calves were living in one pen.

2′-fucosyllactose was supplied to calves via CMR (major components are presented in Table 1) during the whole trial, lasting 6 months. The Start CMR was given from arrival till week 6, which then switched to the Gold Alfa CMR up to day 136. The remaining time calves were fed with the Omega CMR. Solid feed (concentrates) was introduced gradually from arrival, starting with the Avance Alfa up till day 126, then replaced by the Avance Omega till the end of the experiment. Feed was provided ad libitum to calves twice a day (morning and evening) and were daily recorded based on rows (concentrates, straw, corn silage) or groups (CMR) consumption.

TABLE 1
Major components of calf's standard diets in the round 1 trial

| | | | Carbo- | |
|---|---|---|---|---|
| | Protein | Fat | hydrate | Ash |
| Diet | (drywt. %) | (drywt. %) | (drywt. %) | (drywt. %) |
| Start calf milk replacer | 24.1 | 20.8 | 44 | 8.3 |
| Gold Alpha CMR | 18.0 | 18.7 | 54 | 7.4 |
| Gold Omega CMR | 13.7 | 19.2 | 58 | 6.8 |
| Avance Alfa | 16.7 | 4.4 | 57.7 | |
| Avance Omega | 14.6 | 4.4 | 59.5 | |

### Round 2: Rearing Calf

The calves were randomly and gender-equally divided in two groups based on experimental calf milk replacer (CMR): group A (placebo-standard CMR powder of the farm), group B (standard CMR powder supplemented with 0.005% drywt. 2′FL). The calf milk replacer was administered as a liquid to the calves (water was added to the CMR powder to obtain 150 gram CMR powder per liter liquid CMR). Each group involved 100 calves, assigned into four rows (25 calves per row) of two compartment blocks. Each row contained five pens, where four to six calves were living in one pen.

Starting from the day of arrival, calves received standard calf milk replacer (CMR) during a period of 9 weeks. Group A only received the CMR, while the CMR supplied to groups B was supplemented with 0.005% drywt. 2′FL. Calf starter (concentrates) and straw were introduced gradually from day 6 till the end of the experiment (12 weeks) while calves had access to corn silage at weeks 11 and 12. Feed were provided ad libitum to calves twice a day (morning and evening) and were daily recorded based on rows (concentrates, straw, corn silage) or groups (CMR) consumption. Major components of standard CMR and starter diets are presented in table 2.

TABLE 2
Major components of calf's standard diets in the round 2 trial

| | | | Carbo- | Crude | |
|---|---|---|---|---|---|
| | Protein | Fat | hydrate | fiber | Ash |
| | (drywt. | (Drywt. | (Drywt. | (Drywt. | (Drywt. |
| Diet | %) | %) | %) | %) | %) |
| Calf milk | 22 | 18 | 49 | | |
| replacer | Whey powder, vegetable oils (palm, coco, rapeseed), | | | | |
| | hydrolysed wheat gluten, dextrose, premix | | | | |
| Calf | 17.5 | 2.2 | | 5.8 | 6.9 |

| | |
|---|---|
| starter | Corn, soybean meal, beet pulp, barley, wheat, whey |
| | powder, sugar, calcium carbonate, lignocellulose, premix |

### Health, Safety and Performance Observation

Calves were followed up by the farm staffs since arrival till the end of the trial. Fecal consistency in scale 1-4 (Larson et al., 1977, J. Dairy Science, 60 (6): p. 989-991) was recorded daily for the first ten days since arrival. Number of mortalities and signs of adverse effects were registered daily. The animals were treated prophylactically or metaphylactically (herd treatment) with 1 g oxytetracycline 100% (at day 4), 0.5 g Doxylin 50% WSP and 0.8 g Bromhexine 2% (at day 13), and 3.1 ml Tilmicosol 250 mg/ml (at day 24) to prevent and/or treat acute lung inflammation. Individual treatments were followed by remark of the farm staffs and diagnostic of farm veterinary practitioners. Disease incidences and medicinal usage based on individual treatments were recorded to compare between treatment groups. Live weight of the animals was monitored at arrival (d1), two weeks (W2), six weeks (W6), ten weeks (W10) and 12 weeks (W12) and 17 weeks (W17) since arrival. Feed conversion ratio (FCR) of each period was computed based on the input of daily weight gain and daily feed intake.

### Pneumonia Assessment

Acute pneumonia is notified by the farm staff and evaluated by the farm veterinary practitioners based on some or all of the following symptoms (Wolfger et al., 2015, Veterinary Clinics of North America Food Animal 31, 351-365): depression; anorexia; nasal discharge; ocular discharge; cough; increased respiratory rate; increased respiratory noise on chest auscultation; and pyrexia (fever).

### Jejunal Sampling, RNA Extraction and RNAseq (Transcriptomics)

One healthy and middle weight calf of one pen per group was picked for euthanization in the slaughterhouse at day 71 (W11) (8 calves in total, 4 per group). A 20 cm segment of jejunum without Peyer's patch was excised and washed three times with physiological serum using a 30 ml syringe. A small segment (2-3 mm) was further excised and stored in 2 ml RNA protect tissue reagent (Qiagen) containing RNA preservation buffer for transportation at room temperature before long-term storage at −80° C. Jejunal specimens stored in RNA protect tissue reagent were excised further to transfer 50 mg tissue into 2 ml tubes. The tissues were disrupted and homogenized using one stainless steel bead 5 mm in 900 μl QIAzol lysis reagent (QIAgen) in combination with a Fastprep® 24 device. The homogenates were further manipulated according to the guidance of the manufacturer of the RNeasy plus universal tissue mini kit (QIAgen). The total purified RNA were eluted in 40 μl and quickly checked for the concentration and quality using Nanodrop (Implen Nanophotometer®) before storage at −80° C.

Total RNA was submitted to Azenta (Genewiz, Germany) to perform RNA sequencing. The total RNA was checked again for quality and concentration using Qubit and fragment analyzer before the mRNA were selected for polyA library preparation and sequenced using the Illumina® Novaseq™ 2×150 bp technology platform guaranteed 20 million paired-end reads per samples (40 million reads).

RNA sequencing reads were quality-checked with fastqc (https://www.bioinformatics.babraham.ac.uk/projects/fastqc/) and trimmed with Trimmomatic (Bolger et al., 2014, Bioinformatics 30 (15): p. 2114-2120). Reads were aligned to the cow reference (Bos_taurus_UMD_3.1.1) using the genome alignment method in STAR (Dobin et al., 2013, Bioinformatics 29 (1): p. 15-21); Gene expression was quantified by featureCounts (Liao et al., 2014, Bioinformatics 30 (7): p. 923-930) and differential expression analysis was performed using DEseq2 (Love et al., 2014, Genome Biol. 15 (2): 550). Enrichment analysis of differential expressed was done in clusterProfiler (Wu et al., 2021, Innovation 2 (3): 100141) based on Gene Ontology.

### Statistical Analysis

Odds ratios and risk ratios of the pneumonia incidence were evaluated with the epitools R package (https://cran.r-project.org/web/packages/epitools/epitools.pdf). Pneumonia incidence of two rounds of trials was compared between the different treatment groups using Mantel-Haenszel chi-squared test with correction for trial using the epitools R package. Incidence plots were created with the survival R package (https://cran.r-project.org/web/packages/survival/index.html). Differences in mortality were evaluated using the Kaplan-Meier test in the survival package.

### Results

In term of safety, there were no adverse effects from the mammalian milk oligosaccharide as registered by the farm staffs and hence considered safe for the current target animals. In round 1, there were 21 calves dead during 6 months of the trial, of which 12 (4.14%) from group A, and 9 (3.1%) from group B. This ratio is seen normally in most of farms (<5%). There is no significant difference in mortality between the experimental groups. In round 2, during the 12 weeks trial, 7/400 calves died with no significant difference in mortality between the different groups.

Calves mainly got sick in the airway, specifically acute pneumonia (Table 3).

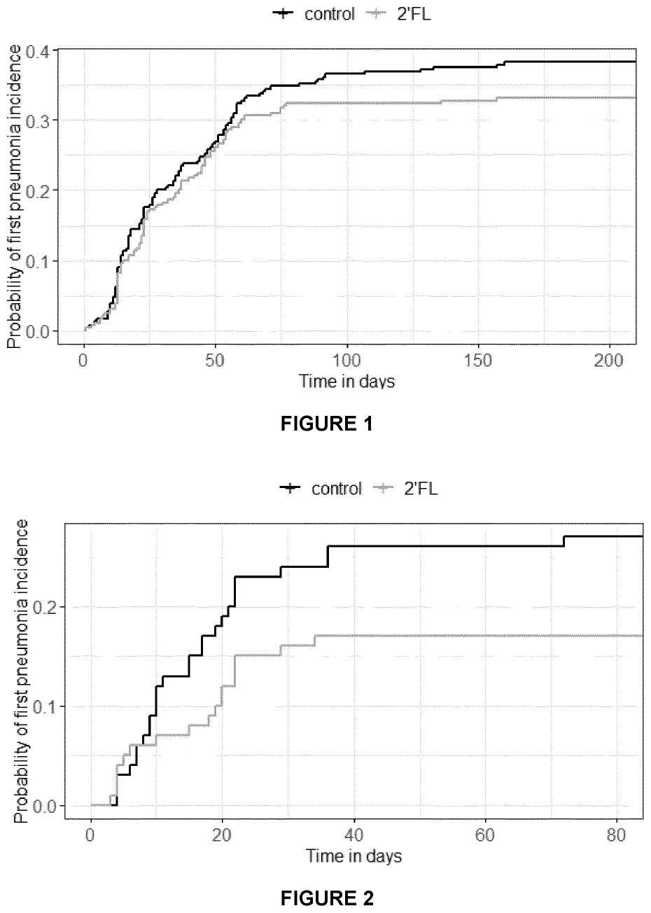

In round 1, number of diagnostic cases with acute pneumonia of the 2′FL-treated group (B; 96 cases out of 290 calves) was lower as compared to the placebo (A; 111 cases out of 290 calves). The relative risk of getting acute pneumonia in group B versus the placebo is 0.86. FIG. 1 depicts the probability of first pneumonia incidence in both groups of the veal calves trial.

In round 2, acute pneumonia affected most of the diseased calves with 27 cases in the placebo (group A; 27%) and 17 cases in the 2′FL-treated group (B; 17%). The relative risk of getting acute pneumonia in group B versus the placebo is 0.63. FIG. 2 shows the probability of first pneumonia incidence in both groups of the rearing calves trial.

When trials in two rounds were integrated for evaluating the efficacy of 2′FL supplementation on pneumonia incidence, the common odds ratio was computed with the value of 1.35 (P-value=0.064). It means that calves that received the standard CMR get acute pneumonia 1.35 times more often than calves that received 2′FL supplemented CMR. This strongly indicates that 2′FL-treated calves were at lower risk getting acute pneumonia as compared to the control.

TABLE 3
Acute pneumonia cases in the trials

| | Placebo | 2′FL | Relative | Common | P- |
|---|---|---|---|---|---|
| Trial | (A) | (B) | risk | odds ratio | value |
| Round 1 (veal calf) | 111 | 96 | 0.86 | 1.35 | 0.064 |
| Round 2 (rearing calf) | 27 | 17 | 0.63 | | |

Following differential expression analysis, enrichment analysis of differential expressed genes (DEGs) and ranked genes were performed. As compared to the placebo, 2′FL-treated group enhanced immune responses in several ways (T cell activation, cell differentiation, cell-cell adhesion, adaptive immune response) (Table 4). Such immune modulation supports the animals combatting against pathogens better, explaining less acute pneumonia in 2′FL-treated group.

TABLE 4
Top 20 gene ontology biological processes up-regulated in the jejunum
of 2′FL-supplemented calves (group B) versus the placebo (group A)

| Description | p.adjust |
|---|---|
| ADAPTIVE_IMMUNE_RESPONSE | 4.15E−06 |
| T_CELL_ACTIVATION | 9.06E−06 |
| MONONUCLEAR_CELL_DIFFERENTIATION | 0.000274 |
| T_CELL_RECEPTOR_COMPLEX | 0.000274 |
| T_CELL_DIFFERENTIATION | 0.000662 |
| EXTERNAL_SIDE_OF_PLASMA_MEMBRANE | 0.000673 |
| NEGATIVE_REGULATION_OF_IMMUNE_SYSTEM_PROCESS | 0.001169 |
| T_CELL_PROLIFERATION | 0.002368 |
| NEGATIVE_REGULATION_OF_ADAPTIVE_IMMUNE_RESPONSE | 0.002368 |
| NEGATIVE_REGULATION_OF_LEUKOCYTE_MEDIATED_IMMUNITY | 0.002368 |
| REGULATION_OF_ADAPTIVE_IMMUNE_RESPONSE | 0.003206 |
| POSITIVE_T_CELL_SELECTION | 0.003206 |
| LEUKOCYTE_DIFFERENTIATION | 0.003462 |
| HETEROTYPIC_CELL_CELL_ADHESION | 0.003478 |
| LYMPHOCYTE_MEDIATED_IMMUNITY | 0.003478 |
| REGULATION_OF_LEUKOCYTE_MEDIATED_IMMUNITY | 0.003478 |
| REGULATION_OF_T_CELL_ACTIVATION | 0.003668 |
| NEGATIVE_REGULATION_OF_LYMPHOCYTE_MEDIATED_IMMUNITY | 0.00552 |
| POSITIVE_THYMIC_T_CELL_SELECTION | 0.00573 |
| ADAPTIVE_IMMUNE_RESPONSE_BASED_ON_SOMATIC_RECOMBINATION_OF_{—} | 0.00573 |
| IMMUNE_RECEPTORS_BUILT_FROM_IMMUNOGLOBULIN_SUPERFAMILY_DOMAINS | |

1.-19. (canceled)

20. A method for preventing or treating pneumonia in a farmed or domesticated animal, the method comprising:
administering a fucosylated mammalian milk oligosaccharide (MMO) to the animal.

21. The method according to claim 20, wherein the fucosylated MMO is administered in an amount sufficient to enhance the T-lymphocyte immunity in the animal.

22. The method according to claim 20, wherein the fucosylated MMO comprises a fucose that is linked to a monosaccharide in an α-1,2-linkage, and wherein the monosaccharide is galactose.

23. The method according to claim 20, wherein the A fucosylated MMO is 2′-fucosyllactose (2′FL).

24. The method according to claim 20, wherein the method does not comprise administering 6′sialyllactose.

25. The method according to claim 20, wherein the method does not comprise administering a sialylated MMO.

26. The method according to claim 20, wherein the method does not comprise administering an additional MMO.

27. The method according to claim 20, wherein the animal is a non-adult animal.

28. The method according to claim 20, wherein the pneumonia is acute pneumonia.

29. The method according to claim 20, wherein the animal is selected from the group consisting of bovine, pig, sheep, goat, horse, donkey, mule, hinny, camelid, dog, cat, rabbit, and poultry.

30. The method according to claim 20, wherein the fucosylated MMO is administered to the animal at least once between birth (0 weeks) and 5 weeks of age.

31. The method according to claim 20, wherein the fucosylated MMO is administered at a daily dose of 0.01-1.0 g.

32. The method according to claim 21, wherein the fucosylated MMO is administered at a daily dose of 0.015-0.10 g.

33. The method according to claim 31, wherein the animal is a bovine or a horse.

34. The method according to claim 20, wherein the fucosylated MMO is administered at a daily dose of 0.01-5.0 g.

35. The method according to claim 34, wherein the fucosylated MMO is administered at a daily dose of 0.5-1.2 g.

36. The method according to claim 34, wherein the animal is a pig, a sheep, a goat, or a camelid.

37. The method according to claim 20, wherein the fucosylated MMO is part of a composition.

38. The method according to claim 37, wherein the composition is a formulated as a pharmaceutical composition, which optionally further comprises a pharmaceutically acceptable carrier, filler, preservative, solubilizer, diluent, excipient, salt, adjuvant, and/or solvent.

39. The method according to claim 37, wherein the composition is a nutritional composition, optionally selected from the group consisting of a milk replacer, creep feed, prestarter diet, starter diet, pre-weaning feed, weaning feed, and post-weaning feed.
